(19) United States
(12) Patent Application Publication
(10) Pub. No.: US 2018/0071380 A1
(43) Pub. Date: Mar. 15, 2018

(54) IMMUNOGENIC COMPOSITIONS FOR USE IN VACCINATION AGAINST BORDETELLA

(71) Applicants: THE REGENTS OF THE UNIVERSITY OF MICHIGAN, ANN ARBOR, MI (US); NANOBIO CORPORATION, ANN ARBOR, MI (US)

(72) Inventors: Paul Makidon, Ann Arbor, MI (US); Vira Bitko, Ann Arbor, MI (US); Douglas Smith, Ann Arbor, MI (US); Ali Fattom, Ann Arbor, MI (US)

(73) Assignees: THE REGENTS OF THE UNIVERSITY OF MICHIGAN, ANN ARBOR, MI (US); NANOBIO CORPORATION, ANN ARBOR, MI (US)

(21) Appl. No.: 15/560,057

(22) PCT Filed: Mar. 18, 2016
(86) PCT No.: PCT/US16/23160

§ 371 Date: Sep. 20, 2017

Related U.S. Application Data
(60) Provisional application No. 62/136,060, filed on Mar. 20, 2015.

(51) Int. Cl. A61K 39/02; A61K 39/39; A61K 47/10

(52) U.S. Cl. CPC A61K 39/099; A61K 2039/57; A61K 47/10; A61K 39/39

(57) ABSTRACT

The present application relates to immunogenic compositions comprising a mixture of Bordetella (e.g., B. pertussis) antigens and an oil in water nanoemulsion. In particular, the invention provides immunogenic compositions comprising nanoemulsion and a combination of Bordetella (e.g., B. pertussis) antigens that have different functions, for example, combinations including B. pertussis adherence factors (adhesins), B. pertussis toxins or B. pertussis virulence factors. Vaccines, methods of treatment, uses of and processes to make a pertussis or whooping cough vaccine are also described. Compositions and methods of the present invention find use in, among other things, clinical (e.g. therapeutic and preventative medicine (e.g., vaccination)) and research applications.

### FIELD OF THE INVENTION

The present invention relates to the field of Bordetella (e.g., B. pertussis) immunogenic compositions and vaccines, their manufacture and the use of such compositions in medicine. More particularly, it relates to vaccine compositions comprising a combination of antigens for the treatment or prevention of Bordetella (e.g., B. pertussis) infection. Methods of using such vaccines in medicine and methods for their preparation are also provided.

### BACKGROUND

Immunization is a principal feature for improving the health of people. Despite the availability of a variety of successful vaccines against many common illnesses, infectious diseases remain a leading cause of health problems and death.

For example, Bordetella pertussis, a gram-negative coccobacillus, is the causative agent of pertussis or whooping cough. Prior to widespread vaccination, pertussis caused up to 13% of all cause childhood mortality. Pertussis infection and pertussis related deaths were reduced dramatically after the introduction of the whole-cell vaccine during the 1950s. The whole cell vaccine (wP) had unwanted side effects that included fever and local reactions, and did not provide consistent protection. An acellular pertussis vaccine (aP) was developed in the 1980s and has now replaced (wP) in major industrialized countries around the world. Acellular pertussis vaccines have historically been effective in protecting infants from developing severe pertussis, but the protection is dramatically reduced within 5-10 years without boosting. However, despite widespread use of acellular vaccines, pertussis has re-emerged since the 1990s and is now estimated to infect 40 million people each year, resulting in approximately 195 000 deaths worldwide, mainly in children. In the US, 48,000 cases were reported in 2012 (50-year high) resulting in 20 deaths.

Studies indicate that the lack of a mucosal immune response specific for pertussis, which correlates with the persistence of nasal carriage of the B. pertussis, is an underlying factor fueling the re-emergence of pertussis. Moreover, there is mounting evidence that the acellular vaccine does not effectively reduce the carriage of B. pertussis in the population, which may have led to the emergence of a highly virulent strain (designated P3) that produces higher amounts of pertussis toxin (Ptx) and does not express pertactin (Prn), rendering the acellular vaccine ineffective against the P3 strain and any similar strains in the future. The Center for Biologics Evaluation and Research at the FDA recently conducted pertussis studies in baboons that showed that TH17 and mucosal immunity are critical in preventing carriage and reinfection by B. pertussis.

### SUMMARY OF THE INVENTION

The present application relates to immunogenic compositions comprising a mixture of Bordetella (e.g., B. pertussis) antigens and an oil in water nanoemulsion. In particular, the invention provides immunogenic compositions comprising nanoemulsion and a combination of Bordetella (e.g., B. pertussis) antigens that have different functions, for example, combinations including B. pertussis adherence factors (adhesins), B. pertussis toxins or B. pertussis virulence factors. Vaccines, methods of treatment, uses of and processes to make a pertussis or whooping cough vaccine are also described. Compositions and methods of the present invention find use in, among other things, clinical (e.g. therapeutic and preventative medicine (e.g., vaccination)) and research applications.

In one embodiment, the present invention provides a novel approach for delivering and inducing a protective immune response against B. pertussis infection by combining one or more B. pertussis immunogenic antigens (e.g., adherence factors, toxins and/or virulence factors), or antigenic fragments thereof, with a delivery and immune enhancing oil-in-water nanoemulsion. In one embodiment, an immunogenic composition comprising nanoemulsion and a combination of B. pertussis antigens induces both mucosal as well as systemic immune responses. In one embodiment, an immunogenic composition comprising nanoemulsion and a combination of B. pertussis antigens induces a Th1 immune response, a Th2 immune response, a Th17 immune response, or any combination thereof. In a preferred embodiment, an immunogenic composition comprising nanoemulsion and a combination of B. pertussis antigens administered (e.g., mucosally (e.g., via nasal mucosa)) to a subject induces a robust IL-17 and/or Th-17 type immune response in the subject. While an understanding of a mechanism is not needed to practice the present invention, and while the present invention is not limited to any particular mechanism, in some embodiments, induction of a Th-17 type immune response in a subject limits and/or prevents carriage of Bordetella (e.g., B. pertussis) in the subject whereas use of conventional injected acellular pertussis vaccine fails to induce Th-17 immune response and also fails to prevent carriage. In another embodiment, an immunogenic composition comprising nanoemulsion and a combination of B. pertussis antigens administered (e.g., mucosally (e.g., via nasal mucosa)) to a subject induces a robust Th-1 type response in the subject. In yet another embodiment, an immunogenic composition comprising nanoemulsion and a combination of B. pertussis antigens administered (e.g., mucosally (e.g., via nasal mucosa)) to a subject induces B. pertussis specific neutralizing antibodies in the subject (e.g., that display bactericidal activity equal to or greater than bactericidal activity of antibodies generated via intramuscular administration of conventional acellular pertussis vaccines).

In another embodiment, the invention provides a method of treating (e.g., prophylactically or therapeutically) a subject with an immunogenic composition of the invention in order to protect the subject against infections with B. pertussis (e.g., thereby reducing morbidity associated with infection from B. pertussis). In some embodiments, methods of treating subjects protects the subject against B. pertussis colonization (e.g., prevents a subject administered the immunogenic composition against infection and disease caused by B. pertussis and/or eliminates carriage of B. pertussis in subjects administered the immunogenic composition (e.g., thereby providing herd immunity and/or eliminating B. pertussis from a population of subjects)). In some embodiments, intranasal administration of an immunogenic composition of the invention reduces carriage of B. pertussis. The invention is not limited by the type of subject administered an immunogenic composition of the invention. Indeed, any subject that can be administered an effective amount of an immunogenic composition of the invention (e.g., to induce an immune response specific to B. pertussis in the subject). In one embodiment, the subject is an adult (e.g., of child bearing age). In one embodiment, the adult is a parent, a grandparent or other adult (e.g., a teacher, a daycare provider, a health professional, or other adult) that is physically around and exposed to children on a daily basis. In one embodiment, the subject is not an adult (e.g., a child) that is physically around and exposed to other non-adults/children on a daily basis.

An immunogenic composition comprising nanoemulsion and a combination of B. pertussis antigens of the invention is not limited by the B. pertussis antigens utilized. Indeed, any combination of B. pertussis immunogenic antigens may be used including, but not limited to, combinations of B. pertussis adherence factors (adhesins), B. pertussis toxins, B. pertussis virulence factors, B. pertussis outer-membrane proteins, and/or immunogenic fragments of each of the foregoing. Exemplary B. pertussis immunogenic antigens are described herein and include, but are not limited to, pertussis toxin (Ptx), filamentous hæmagglutinin adhesin (FHA), pertactin (PRN), fimbria (e.g., fimbrial-2 and fimbrial-3), attachment pili, tracheal cytotoxin (TCT), or other B. pertussis immunogenic antigens known in the art. Immunogenic B. pertussis antigens can be from any strain of B. pertussis or any strain of Bordetella that causes respiratory infection (e.g., B. bronchiseptica, B. parapertussis, or B. holmesii). As described in detail herein, an immunogenic B. pertussis antigen may comprise at least one nucleotide modification (e.g., denoting an attenuating phenotype and/or a more immunogenic antigen). In another embodiment, an immunogenic B. pertussis antigen or antigenic fragment thereof is present in a fusion protein. Also described herein, an immunogenic B. pertussis antigen may be configured to be multivalent.

The present invention is not limited by the nanoemulsion utilized in an immunogenic composition comprising nanoemulsion and a combination of B. pertussis antigens. Indeed, any nanoemulsion described herein may be utilized. In one non-limiting example, the nanoemulsion comprises (a) at least one cationic surfactant and at least one non-cationic surfactant; (b) at least one cationic surfactant and at least one non-cationic surfactant, wherein the non-cationic surfactant is a nonionic surfactant; (c) at least one cationic surfactant and at least one non-cationic surfactant, wherein the non-cationic surfactant is a polysorbate nonionic surfactant, a poloxamer nonionic surfactant, or a combination thereof; (d) at least one cationic surfactant and at least one nonionic surfactant which is polysorbate 20, polysorbate 80, poloxamer 188, poloxamer 407, or a combination thereof; (e) at least one cationic surfactant and at least one nonionic surfactant which is polysorbate 20, polysorbate 80, poloxamer 188, poloxamer 407, or a combination thereof, and wherein the nonionic surfactant is present at about 0.01% to about 5.0%, or at about 0.1% to about 3%; (f) at least one cationic surfactant and at least one non-cationic surfactant, wherein the non-cationic surfactant is a nonionic surfactant, and the non-ionic surfactant is present in a concentration of about 0.05% to about 10%, about 0.05% to about 7.0%, about 0.1% to about 7%, or about 0.5% to about 4%; (g) at least one cationic surfactant and at least one a nonionic surfactant, wherein the cationic surfactant is present in a concentration of about 0.05% to about 2% or about 0.01% to about 2%; or (h) any combination thereof.

In a preferred embodiment, an immunogenic composition comprising nanoemulsion and a combination of B. pertussis antigens of the invention comprises droplets having an average diameter of less than about 1000 nm. In one embodiment, the nanoemulsion present in an immunogenic composition comprising nanoemulsion and a combination of B. pertussis antigens comprises: (a) an aqueous phase, (b) at least one oil, (c) at least one surfactant, (d) at least one organic solvent, and (e) optionally at least one chelating agent. Preferably the B. pertussis antigens are present in the nanoemulsion droplets. In another embodiment, an immunogenic composition comprising nanoemulsion and a combination of B. pertussis antigens is administered intranasally. As described herein, additional components may be added to an immunogenic composition comprising nanoemulsion and a combination of B. pertussis antigens including, but not limited to, one or more additional adjuvants described herein.

In one embodiment, an immunogenic composition comprising nanoemulsion and a combination of B. pertussis antigens is formulated into any pharmaceutically acceptable dosage form, such as a liquid dispersion, gel, aerosol, pulmonary aerosol, nasal aerosol, ointment, cream, or solid dose. In a further embodiment, an immunogenic composition comprising nanoemulsion and a combination of B. pertussis antigens is not systemically toxic to the subject, and produces minimal or no inflammation upon administration. In another embodiment, the subject undergoes seroconversion after a single administration of the immunogenic composition. In a further embodiment, an immunogenic composition comprising nanoemulsion and a combination of B. pertussis antigens is formulated as a liquid dispersion, gel, aerosol, pulmonary aerosol, nasal aerosol, ointment, cream, or solid dose. In addition, an immunogenic composition comprising nanoemulsion and a combination of B. pertussis antigens may be administered via any pharmaceutically acceptable method, such as parenterally, orally, intranasally, or rectally. The parenteral administration can be by intradermal, subcutaneous, intraperitoneal or intramuscular injection.

In one embodiment, the invention provides a method for generating an B. pertussis specific immune response in a subject (e.g., thereby enhancing immunity to B. pertussis infection in the subject) comprising administering to the subject an immunogenic composition comprising nanoemulsion and a combination of B. pertussis antigens described herein. Another embodiment of the invention is directed to a method for inhibiting signs, symptoms and/or conditions of B. pertussis infection and/or disease in a subject comprising the step of administering to the subject an effective amount of an immunogenic composition comprising nanoemulsion and a combination of B. pertussis antigens according to the invention. In one embodiment, the subject produces a seroprotective immune response after at least a single administration of the immunogenic composition. In one embodiment, a seroprotective immune response (e.g., comprising both mucosal and systemic B. pertussis specific antibodies and/or B. pertussis specific cellular immune responses (e.g., Th-17 and/or Th-1 immune responses) induced after administration to a subject is effective against one or more strains of B. pertussis (e.g., is cross-reactive with other strains).

In another embodiment, the invention provides a method of preventing and/or treating infection and/or disease caused by a species of Bordetella (e.g., B. pertussis (e.g., whooping cough)) comprising administering an effective amount of an immunogenic composition of the invention to a subject. In another embodiment, the invention provides the use of an immunogenic composition of the invention for the manufacture of a medicament (e.g., a vaccine) for the treatment of Bordetella (e.g., B. pertussis) infection (e.g., whooping cough). In still another embodiment, the invention provides an immunogenic composition (e.g., any one of the immunogenic compositions of the invention) for use in the treatment of Bordetella (e.g., B. pertussis) infection.

### DESCRIPTION OF THE DRAWINGS

FIG. 1 shows antibody levels for (A) pertussis toxin, (B) FHA and (C) Pertactin upon either intranasal NE-aP vaccination or intramuscular alum-aP IM vaccination, as assessed by ELISA.

FIG. 2 shows bactericidal activity in the sera of vaccinated rats six weeks after the third immunization, shown as a percent of CFU reduction compared to negative sera control samples.

FIG. 3 shows secretion of cytokine IL-17 by peripheral blood mononuclear cells (PBMCs) after re-stimulation against each vaccine antigen, following (A) intranasal NE-aP vaccination, (B) intramuscular alum-aP IM vaccination, and (C) PBS control.

FIG. 4 shows secretion of cytokines IL-5 (FIG. 4A) and INF-γ (FIG. 4B) by PBMCs after re-stimulation against each vaccine antigen, following intranasal NE-aP vaccination (IN), intramuscular alum-aP IM vaccination (IM), or PBS control (PBS).

### DEFINITIONS

To facilitate an understanding of the present invention, a number of terms and phrases are defined below:

As used herein the term “microorganism” refers to microscopic organisms and taxonomically related macroscopic organisms within the categories of algae, bacteria, fungi (including lichens), protozoa, viruses, and subviral agents. The term microorganism encompasses both those organisms that are in and of themselves pathogenic to another organism (e.g., animals, including humans, and plants) and those organisms that produce agents that are pathogenic to another organism, while the organism itself is not directly pathogenic or infective to the other organism. As used herein the term “pathogen,” and grammatical equivalents, refers to an organism, including microorganisms, that causes disease in another organism (e.g., animals and plants) by directly infecting the other organism, or by producing agents that causes disease in another organism (e.g., bacteria that produce pathogenic toxins and the like).

As used herein the term “disease” refers to a deviation from the condition regarded as normal or average for members of a species or group, and which is detrimental to an affected individual under conditions that are not inimical to the majority of individuals of that species or group (e.g., diarrhea, nausea, fever, pain, and inflammation etc.). A disease may be caused or result from contact by microorganisms and/or pathogens.

The ability of an immunogenic composition (e.g., vaccine) of the invention to protect against Bordetella (e.g., B. pertussis) colonization, as provided herein, means that the active components of the immunogenic composition (e.g., the nanoemulsion plus Bordetella antigens) may protect against disease not only in an immunized host but also, by eliminating carriage among immunized individuals, the pathogen and any disease it causes may be eliminated from the population as a whole (e.g., herd immunity).

The terms “host” or “subject,” as used herein, are used interchangeably to refer to organisms to be treated by the compositions and methods of the present invention. Such organisms include organisms that are exposed to, or suspected of being exposed to, one or more pathogens (e.g., B. pertussis). Such organisms also include organisms to be treated so as to prevent undesired exposure to pathogens. Organisms include, but are not limited to animals (e.g., humans, domesticated animal species, wild animals).

As used herein, the term “inactivating,” and grammatical equivalents, means having the ability to kill, eliminate or reduce the capacity of a pathogen to infect and/or cause a pathological responses in a host.

As used herein, the term “fusigenic” is intended to refer to an emulsion that is capable of fusing with the membrane of a microbial agent (e.g., a bacterium or bacterial spore). Specific examples of fusigenic emulsions include, but are not limited to, W₈₀8P described in U.S. Pat. Nos. 5,618,840; 5,547,677; and 5,549,901 and NP9 described in U.S. Pat. No. 5,700,679, each of which is herein incorporated by reference in their entireties. NP9 is a branched poly (oxy-1,2 ethaneolyl),alpha-(4-nonylphenal)-omega-hydroxy-surfactant. While not being limited to the following, NP9 and other surfactants that may be useful in the present invention are described in Table 1 of U.S. Pat. No. 5,662,957, herein incorporated by reference in its entirety.

As used herein, the term “lysogenic” refers to an emulsion that is capable of disrupting the membrane of a microbial agent (e.g., a bacterium or bacterial spore). In preferred embodiments of the present invention, the presence of both a lysogenic and a fusigenic agent in the same composition produces an enhanced inactivating effect than either agent alone. Methods and compositions (e.g., vaccines) using this improved antimicrobial composition are described in detail herein.

The term “nanoemulsion,” as used herein, includes small oil-in-water dispersions or droplets, as well as other lipid structures which can form as a result of hydrophobic forces which drive apolar residues (i.e., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. The present invention contemplates that one skilled in the art will appreciate this distinction when necessary for understanding the specific embodiments herein disclosed. The terms “emulsion” and “nanoemulsion” are often used herein, interchangeably, to refer to the nanoemulsions of the present invention.

The term “surfactant” refers to any molecule having both a polar head group, which energetically prefers solvation by water, and a hydrophobic tail that is not well solvated by water. The term “cationic surfactant” refers to a surfactant with a cationic head group. The term “anionic surfactant” refers to a surfactant with an anionic head group.

The terms “Hydrophile-Lipophile Balance Index Number” and “HLB Index Number” refer to an index for correlating the chemical structure of surfactant molecules with their surface activity. The HLB Index Number may be calculated by a variety of empirical formulas as described by Meyers, (Meyers, Surfactant Science and Technology, VCH Publishers Inc., New York, pp. 231-245 [1992]), incorporated herein by reference. As used herein, the HLB Index Number of a surfactant is the HLB Index Number assigned to that surfactant in McCutcheon's Volume 1: Emulsifiers and Detergents North American Edition, 1996 (incorporated herein by reference). The HLB Index Number ranges from 0 to about 70 or more for commercial surfactants. Hydrophilic surfactants with high solubility in water and solubilizing properties are at the high end of the scale, while surfactants with low solubility in water that are good solubilizers of water in oils are at the low end of the scale.

As used herein, the term “germination enhancers” refer to compounds (e.g., amino acids (e.g., L-amino acids (L-alanine)), CaCl₂, Inosine, nitrogenous bases, etc.) that act, for example, to enhance the germination of certain strains of bacteria.

As used herein the term “interaction enhancers” refers to compounds that act to enhance the interaction of an emulsion with the cell wall of a bacteria (e.g., a Gram negative bacteria). Contemplated interaction enhancers include, but are not limited to, chelating agents (e.g., ethylenediaminetetraacetic acid (EDTA), ethylenebis(oxyethylenenitrilo)tetraacetic acid (EGTA), and the like) and certain biological agents (e.g., bovine serum albumin (BSA) and the like).

The terms “buffer” or “buffering agents” refer to materials, that when added to a solution, cause the solution to resist changes in pH.

The terms “reducing agent” and “electron donor” refer to a material that donates electrons to a second material to reduce the oxidation state of one or more of the second material's atoms.

The term “monovalent salt” refers to any salt in which the metal (e.g., Na, K, or Li) has a net 1+ charge in solution (i.e., one more proton than electron).

The term “divalent salt” refers to any salt in which a metal (e.g., Mg, Ca, or Sr) has a net 2+ charge in solution.

The terms “chelator” or “chelating agent” refer to any materials having more than one atom with a lone pair of electrons that are available to bond to a metal ion.

The term “solution” refers to an aqueous or non-aqueous mixture.

As used herein, the term “therapeutic agent,” refers to compositions that decrease the infectivity, morbidity, or onset of mortality in a host contacted by a pathogenic microorganism or that prevent infectivity, morbidity, or onset of mortality in a host contacted by a pathogenic microorganism. Such agents may additionally comprise pharmaceutically acceptable compounds (e.g., adjutants, excipients, stabilizers, diluents, and the like). In some embodiments, the therapeutic agents (e.g., immunogenic compositions or vaccines) of the present invention are administered in the form of topical emulsions, injectable compositions, ingestible solutions, and the like. When the route is topical, the form may be, for example, a spray (e.g., a nasal spray).

As used herein, the term “topically active agents” refers to compositions of the present invention that illicit a pharmacological response at the site of application (contact) to a host.

As used herein, the term “systemically active drugs” is used broadly to indicate a substance or composition that will produce a pharmacological response at a site remote from the point of application or entry into a subject.

As used herein, the terms “a composition for inducing an immune response,” “immunogenic composition” or grammatical equivalents refer to a composition that, once administered to a subject (e.g., once, twice, three times or more (e.g., separated by weeks, months or years)), stimulates, generates and/or elicits an immune response in the subject (e.g., resulting in total or partial immunity to a microorganism (e.g., pathogen) capable of causing disease). In preferred embodiments of the invention, the composition comprises a nanoemulsion and an immunogen. In further preferred embodiments, the composition comprising a nanoemulsion and an immunogen comprises one or more other compounds or agents including, but not limited to, therapeutic agents, physiologically tolerable liquids, gels, carriers, diluents, adjuvants, excipients, salicylates, steroids, immunosuppressants, immunostimulants, antibodies, cytokines, antibiotics, binders, fillers, preservatives, stabilizing agents, emulsifiers, and/or buffers. A composition for inducing an immune response (e.g., immunogenic composition of the invention) may be administered to a subject as a vaccine (e.g., to prevent or attenuate a disease (e.g., by providing to the subject total or partial immunity against the disease or the total or partial attenuation (e.g., suppression) of a sign, symptom or condition of the disease).

As used herein, the term “adjuvant” refers to any substance that can stimulate an immune response (e.g., a mucosal immune response). Some adjuvants can cause activation of a cell of the immune system (e.g., an adjuvant can cause an immune cell to produce and secrete a cytokine). Examples of adjuvants that can cause activation of a cell of the immune system include, but are not limited to, saponins purified from the bark of the Q. saponaria tree, such as QS21 (a glycolipid that elutes in the 21st peak with HPLC fractionation; Aquila Biopharmaceuticals, Inc., Worcester, Mass.); poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). Traditional adjuvants are well known in the art and include, for example, aluminum phosphate or hydroxide salts (“alum”). In some embodiments, compositions of the present invention (e.g., comprising nanoemulsion inactivated RSV) are administered with one or more adjuvants (e.g., to skew the immune response towards a Th1 or Th2 type response).

As used herein, the term “an amount effective to induce an immune response” (e.g., of a composition for inducing an immune response), refers to the dosage level required (e.g., when administered to a subject) to stimulate, generate and/or elicit an immune response in the subject. An effective amount can be administered in one or more administrations (e.g., via the same or different route), applications or dosages and is not intended to be limited to a particular formulation or administration route.

As used herein, the term “under conditions such that said subject generates an immune response” refers to any qualitative or quantitative induction, generation, and/or stimulation of an immune response (e.g., innate or acquired).

A used herein, the term “immune response” refers to a response by the immune system of a subject. For example, immune responses include, but are not limited to, a detectable alteration (e.g., increase) in Toll receptor activation, lymphokine (e.g., cytokine (e.g., Th1, Th17, or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the term “immunity” refers to protection from disease (e.g., preventing or attenuating (e.g., suppression) of a sign, symptom or condition of the disease) upon exposure to a microorganism (e.g., pathogen) capable of causing the disease. Immunity can be innate (e.g., non-adaptive (e.g., non-acquired) immune responses that exist in the absence of a previous exposure to an antigen) and/or acquired (e.g., immune responses that are mediated by B and T cells following a previous exposure to antigen (e.g., that exhibit increased specificity and reactivity to the antigen)).

As used herein, the terms “antigen” and “immunogen” are used interchangeably to refer to proteins, polypeptides, glycoproteins or derivatives or fragment that can contain one or more epitopes (linear, conformation, sequential, T-cell) which can elicit an immune response. In preferred embodiments, immunogens/antigens elicit immunity against the immunogen/antigen (e.g., a pathogen or a pathogen product) when administered in combination with a nanoemulsion of the present invention.

The term “antigenic fragment,” for example, an antigenic fragment of pertussis toxin, refers to a peptide having at least about 5 consecutive amino acids of a naturally occurring or mutant pertussis toxin protein, or if used to describe an antigenic fragment of a different antigen refers to a peptide having at least about 5 consecutive amino acids of a naturally occurring or mutant version of the antigen. An antigenic fragment can be any suitable length, such as between about 5 amino acids in length up to and including full length protein. For example, an antigenic fragment can be about 10%, about 15%, about 20%, about 30%, about 40%, about 50%, about 60%, about 70%, about 80%, about 90%, or about 100% of the full length of the native protein.

As used herein, the term “pathogen product” refers to any component or product derived from a pathogen including, but not limited to, polypeptides, peptides, proteins, nucleic acids, membrane fractions, and polysaccharides.

As used herein, the term “enhanced immunity” refers to an increase in the level of acquired immunity to a given pathogen following administration of a vaccine of the present invention relative to the level of acquired immunity when a vaccine of the present invention has not been administered.

As used herein, the terms “purified” or “to purify” refer to the removal of contaminants or undesired compounds from a sample or composition. As used herein, the term “substantially purified” refers to the removal of from about 70 to 90%, up to 100%, of the contaminants or undesired compounds from a sample or composition.

As used herein, the term “isolated” refers to proteins, glycoproteins, peptide derivatives or fragment or polynucleotide that is independent from its natural location. Bacterial (e.g., B. pertussis) components that are independently obtained through recombinant genetics means typically leads to products that are relatively purified.

As used herein, the term “surface” is used in its broadest sense. In one sense, the term refers to the outermost boundaries of an organism or inanimate object (e.g., vehicles, buildings, and food processing equipment, etc.) that are capable of being contacted by the compositions of the present invention (e.g., for animals: the skin, hair, and fur, etc., and for plants: the leaves, stems, flowering parts, and fruiting bodies, etc.). In another sense, the term also refers to the inner membranes and surfaces of animals and plants (e.g., for animals: the digestive tract, vascular tissues, and the like, and for plants: the vascular tissues, etc.) capable of being contacted by compositions by any of a number of transdermal delivery routes (e.g., injection, ingestion, transdermal delivery, inhalation, and the like).

As used herein, the term “sample” is used in its broadest sense. In one sense it can refer to animal cells or tissues. In another sense, it is meant to include a specimen or culture obtained from any source, such as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

As used herein, the terms “administration” and “administering” refer to the act of giving a composition of the present invention (e.g., a composition for inducing an immune response (e.g., a composition comprising a nanoemulsion and an immunogen)) to a subject. Exemplary routes of administration to the human body include, but are not limited to, through the eyes (ophthalmic), mouth (oral), skin (transdermal), nose (nasal), lungs (inhalant), oral mucosa (buccal), ear, rectal, by injection (e.g., intravenously, subcutaneously, intraperitoneally, etc.), topically, and the like.

As used herein, the terms “co-administration” and “co-administering” refer to the administration of at least two agent(s) (e.g., a composition comprising a nanoemulsion and an immunogen and one or more other agents—e.g., an adjuvant) or therapies to a subject. In some embodiments, the co-administration of two or more agents or therapies is concurrent. In other embodiments, a first agent/therapy is administered prior to a second agent/therapy. In some embodiments, co-administration can be via the same or different route of administration. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent. In other embodiments, co-administration is preferable to elicit an immune response in a subject to two or more different immunogens (e.g., microorganisms (e.g., pathogens)) at or near the same time (e.g., when a subject is unlikely to be available for subsequent administration of a second, third, or more composition for inducing an immune response).

As used herein, the term “topically” refers to application of a compositions of the present invention (e.g., a composition comprising a nanoemulsion and an immunogen) to the surface of the skin and/or mucosal cells and tissues (e.g., alveolar, buccal, lingual, masticatory, vaginal or nasal mucosa, and other tissues and cells which line hollow organs or body cavities).

In some embodiments, the compositions of the present invention are administered in the form of topical emulsions, injectable compositions, ingestible solutions, and the like. When the route is topical, the form may be, for example, a spray (e.g., a nasal spray), a cream, or other viscous solution (e.g., a composition comprising a nanoemulsion and an immunogen in polyethylene glycol).

The terms “pharmaceutically acceptable” or “pharmacologically acceptable,” as used herein, refer to compositions that do not substantially produce adverse reactions (e.g., toxic, allergic or immunological reactions) when administered to a subject. As used herein, the term “pharmaceutically acceptable carrier” refers to any of the standard pharmaceutical carriers including, but not limited to, phosphate buffered saline solution, water, and various types of wetting agents (e.g., sodium lauryl sulfate), any and all solvents, dispersion media, coatings, sodium lauryl sulfate, isotonic and absorption delaying agents, disintrigrants (e.g., potato starch or sodium starch glycolate), polyethylethe glycol, and the like. The compositions also can include stabilizers and preservatives. Examples of carriers, stabilizers and adjuvants have been described and are known in the art (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a composition of the present invention that is physiologically tolerated in the target subject. “Salts” of the compositions of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compositions of the invention and their pharmaceutically acceptable acid addition salts. Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like. Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁻, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound. For therapeutic use, salts of the compositions of the present invention are contemplated as being pharmaceutically acceptable. However, salts of acids and bases that are non-pharmaceutically acceptable may also find use, for example, in the preparation or purification of a pharmaceutically acceptable composition.

As used herein, the term “at risk for disease” refers to a subject that is predisposed to experiencing a particular disease. This predisposition may be genetic (e.g., a particular genetic tendency to experience the disease, such as heritable disorders), or due to other factors (e.g., age, environmental conditions, exposures to detrimental compounds present in the environment, etc.). Thus, it is not intended that the present invention be limited to any particular risk (e.g., a subject may be “at risk for disease” simply by being exposed to and interacting with other people), nor is it intended that the present invention be limited to any particular disease.

“Nasal application”, as used herein, means applied through the nose into the nasal or sinus passages or both. The application may, for example, be done by drops, sprays, mists, coatings or mixtures thereof applied to the nasal and sinus passages.

As used herein, the term “kit” refers to any delivery system for delivering materials. In the context of immunogenic agents (e.g., compositions comprising a nanoemulsion and an immunogen), such delivery systems include systems that allow for the storage, transport, or delivery of immunogenic agents and/or supporting materials (e.g., written instructions for using the materials, etc.) from one location to another. For example, kits include one or more enclosures (e.g., boxes) containing the relevant immunogenic agents (e.g., nanoemulsions) and/or supporting materials. As used herein, the term “fragmented kit” refers to delivery systems comprising two or more separate containers that each contain a subportion of the total kit components. The containers may be delivered to the intended recipient together or separately. For example, a first container may contain a composition comprising a nanoemulsion and an immunogen for a particular use, while a second container contains a second agent (e.g., an antibiotic or spray applicator). Indeed, any delivery system comprising two or more separate containers that each contains a subportion of the total kit components are included in the term “fragmented kit.” In contrast, a “combined kit” refers to a delivery system containing all of the components of an immunogenic agent needed for a particular use in a single container (e.g., in a single box housing each of the desired components). The term “kit” includes both fragmented and combined kits.

### DESCRIPTION OF THE INVENTION

The present invention relates to immunogenic compositions comprising a mixture of Bordetella pertussis antigens and an oil in water nanoemulsion. In particular, the invention provides immunogenic compositions comprising nanoemulsion and a combination of B. pertussis antigens that have different functions, for example, combinations including a B. pertussis adherence factors (adhesins), B. pertussis toxins or B. pertussis virulence factors. Vaccines, methods of treatment, uses of and processes to make a pertussis or whooping cough vaccine are also described. Compositions and methods of the present invention find use in, among other things, clinical (e.g. therapeutic and preventative medicine (e.g., vaccination)) and research applications.

Bordetella pertussis was one of the leading causes of childhood mortality prior to the introduction of the whole-cell vaccine in the 1950s. The whole-cell vaccine reduced pertussis infection and related deaths incidence dramatically but showed inconsistency, and raised concerns regarding safety. The acellular pertussis vaccine was introduced in the 1990s, and showed consistency and efficacy that led most of the developed world to adopt it. However, pertussis re-emerged soon after the adoption of the acellular vaccine and is now estimated to infect 40 million people each year, leading to 195,000 deaths worldwide, mainly in children. Research has been conducted into the probable cause for the reemergence of pertussis, and a breakthrough came through the development of the baboon animal model in the FDA laboratories which closely resembles the human disease. Warfel et al. demonstrated that the acellular vaccine protected from pertussis disease and elicited a strong immune response, but failed to reduce carriage of B. pertussis. Baboons vaccinated with the acellular vaccine performed similarly to non-vaccinated baboons in clearing the bacteria over 35 days. In contrast, the whole cell vaccine prevented pertussis disease and cleared the organism within 18 days. Convalescent animals did not show any nasal carriage. However, the acellular vaccinated animals that showed no sign of the disease did in fact transmit B. pertussis to naïve animals, indicating that these animals, while not manifesting infection, acted to transmit B. pertussis (e.g., carriage of B. pertussis occurred in the acellular vaccinated subjects). Warfel et al. further characterized the different T-cell memory responses induced via the different vaccines: Th1, Th2, and Th17 using IFNγ as an indicator of Th1 response, IL-5 as an indicator of Th2 response, and IL-17 for the Th17 response. While the acellular vaccine induced a Th2 response with a weaker Th1 response (strong IL-5 and a weak IFNγ), the whole cell vaccine induced a strong Th1 and Th17 responses (IFNγ and IL-17), thus resembling the natural immunity seen in the convalescent animals that were protected against disease and nasal carriage. Th-17 has been identified for its protective role in host defense against a number of viral and bacterial pathogens at epithelial and mucosal surfaces.

Pertussis infection progresses through several different clinical stages. The incubation period of pertussis is commonly 7-10 days, with a range of 4-21 days, and rarely may be as long as 42 days. The clinical course of the illness is divided into three stages. The first stage, the catarrhal stage, is characterized by the insidious onset of coryza (runny nose), sneezing, low-grade fever, and a mild, occasional cough, similar to the common cold. The cough gradually becomes more severe, and after 1-2 weeks, the second, or paroxysmal stage, begins. Fever is generally minimal throughout the course of the illness. It is during the paroxysmal stage that the diagnosis of pertussis is usually suspected. Characteristically, the patient has bursts, or paroxysms, of numerous, rapid coughs, apparently due to difficulty expelling thick mucus from the tracheobronchial tree. At the end of the paroxysm, a long inspiratory effort is usually accompanied by a characteristic high-pitched whoop. During such an attack, a patient may become cyanotic (turn blue). Children and young infants, especially, appear very ill and distressed. Vomiting and exhaustion commonly follow the episode. The person does not appear to be ill between attacks. Paroxysmal attacks occur more frequently at night, with an average of 15 attacks per 24 hours. During the first 1 or 2 weeks of this stage, the attacks increase in frequency, remain at the same level for 2 to 3 weeks, and then gradually decrease. The paroxysmal stage usually lasts 1 to 6 weeks but may persist for up to 10 weeks. Infants younger than 6 months of age may not have the strength to have a whoop, but they do have paroxysms of coughing. In the convalescent stage, recovery is gradual. The cough becomes less paroxysmal and disappears in 2 to 3 weeks. However, paroxysms often recur with subsequent respiratory infections for many months after the onset of pertussis.

Adolescents, adults and children partially protected by the vaccine may become infected with B. pertussis but may have milder disease than infants and young children. Pertussis infection in these persons may be asymptomatic, or present as illness ranging from a mild cough illness to classic pertussis with persistent cough (e.g., lasting more than 7 days).

Even though the disease may be milder in older persons, those who are infected may transmit the disease to other susceptible persons (e.g., babies, infants, young children, immune compromised or unimmunized or incompletely immunized infants). Older persons are often found to have the first case in a household with multiple pertussis cases, and are often the source of infection for children.

As described herein, experiments were conducted during development of embodiments of the invention in order to determine if a new immunogenic composition comprising nanoemulsion and one or more B. pertussis antigens could be generated and used in a method of inducing B. pertussis specific immune responses in a subject. As described Example 1, experiments were conducted wherein rats were administered an immunogenic composition of the invention intranasally with immunogenicity and bactericidal activity subsequently assessed. The immunogenic composition of the invention was compared with a convention acellular pertussis vaccine administered intramuscularly as a positive control. Intranasal vaccination with the immunogenic composition of the invention elicited high levels of antibody (measured by ELISA) against all three components of the vaccine (See Example 1). In addition, sera from vaccinated animals were tested for bactericidal activity at six weeks after the third dose, as an immunological correlate of vaccine protection. Animals vaccinated intranasally with the immunogenic composition of the invention showed a significantly high level of bactericidal activity despite somewhat lower levels of antibodies compared to the positive control intramuscular vaccine (See Example 1). The NE adjuvant enabled intranasal immunization and elicitation of immune response with high levels of bactericidal activity equivalent to or stronger than a conventional acellular pertussis vaccine administered intramuscularly that served as an immunological correlate and predictor of a vaccine protection. Furthermore, LUMINEX multiplex analysis was used to evaluate mucosal immunity elicited by the immunogenic composition of the invention in rats and the results indicated that a strong IL-17 response was elicited against FHA, pertussis toxin, and to a somewhat lesser extent against pertactin. In sharp contrast, the conventional vaccine administered intramuscularly elicited a low to negligible IL-17 response (See Example 1).

Accordingly, in one embodiment, the invention provides immunogenic compositions and methods of using the same to induce systemic, pertussis specific immune responses (e.g., systemic immunity) and to elicit a pertussis specific IL-17 response. Such methods are achievable utilizing intranasal delivery of immunogenic compositions of the invention. While an understanding of a mechanism is not needed to practice the present invention, and while the present invention is not limited to any particular mechanism of action, in one embodiment, administration of an immunogenic composition of the invention at or close to the site of colonization participates in conferring systemic immunity and protecting against colonization and transmission of B. pertussis. Accordingly, in one embodiment, use of the compositions and methods disclosed herein are utilized for intranasal administration and to confer mucosal immunity to B. pertussis, to prevent colonization and transmission, and restore herd immunity against pertussis.

The B. pertussis infection life cycle involves commensal colonization whereby the bacteria attach to ciliated airway epithelium, initiation of infection by accessing adjoining tissues or the bloodstream, anaerobic multiplication in the blood, interplay between B. pertussis virulence factors/determinants and the host defense mechanisms, and induction of complications associated with B. pertussis infection including cough, fever, breathing complications, bronchopneumonia, vomiting, exhaustion and/or other B. pertussis related morbidity.

B. pertussis antigens involved throughout infection are described herein. Different molecules on the surface of the B. pertussis are involved in different steps of the infection cycle. By targeting the immune response against an effective amount of a combination of particular antigens involved in different processes of B. pertussis infection, an immunogenic composition comprising nanoemulsion and a combination of B. pertussis antigens is achieved.

In particular, combinations of certain antigens from different classes, some of which are involved in adhesion to host cells, some of which are involved in transporter functions, some of which are toxins or regulators of virulence and immunodominant antigens can elicit an immune response which protects against multiple stages of infection.

The effectiveness of the immune response can be measured in both research and clinical settings for example, in animal model assays and/or using an opsonophagocytic assay).

An additional advantage of the invention is that the combination of antigens of the invention from different families of proteins in an immunogenic composition enables protection against a variety of different strains.

In one embodiment, the invention relates to immunogenic compositions comprising a plurality of proteins selected from at least two different categories of protein, having different functions within B. pertussis. Examples of such categories of proteins are extracellular binding proteins, transporter proteins, metabolic proteins, toxins or regulators of virulence and other immunodominant proteins. The vaccine combinations of the invention are effective against homologous B. pertussis strains (strains from which the antigens are derived) and preferably also against heterologous B. pertussis strains.

An immunogenic composition of the invention comprises a number of proteins equal to or greater than 2, 3, 4, 5 or 6 selected from 2 or 3 of the following groups:
group a)—at least one B. pertussis extracellular component binding protein or immunogenic fragment thereof selected from filamentous hæmagglutinin adhesin (FHA) and/or fimbriae;
group b)—at least one B. pertussis transporter protein (autotransporter proteins) or immunogenic fragment thereof selected from pertactin (PRN), Vag8, BrkA, SphB1, and/or Tracheal colonization factor (TcfA); and
group c)—at least one B. pertussis regulator of virulence, toxin or immunogenic fragment thereof selected from pertussis toxin (PT), adenylate cyclase (CyaA), Type III secretion, dermonectrotic toxin (DNT), Tracheal cytotoxin (TCT), and/or LPS (e.g., wlb locus, wbm locus, PagP).

For example a first protein is selected from group a), b) or c) and a second protein is selected from a group selected from groups a), b) and c) which does not include the second protein.

In a preferred embodiment, the immunogenic composition of the invention contains at least one protein selected from group a) and an additional protein selected from group b) and/or group c).

In a further embodiment, the immunogenic composition of the invention contains at least one antigen selected from group b) and an additional protein selected from group c) and/or group a).

In a further embodiment, the immunogenic composition of the invention contains at least one antigen selected from group c) and an additional protein selected from group a) and/or group b).

The immunogenic composition of the invention may contains proteins from B. pertussis, B. bronchiseptica, B. parapertussis, and/or B. holmesii.

In a further embodiment, the immunogenic composition comprises one or more other B. pertussis proteins or immunogenic fragment thereof selected from flagella, Type IV pili, Capsule, Alcaligin and/or Vrg loci.

Where a protein is specifically mentioned herein, it is preferably a reference to a native or recombinant, full-length protein or optionally a mature protein in which any signal sequence has been removed. The protein may be isolated directly from a Bordetella strain or produced by recombinant DNA techniques. Immunogenic fragments of the protein may be incorporated into the immunogenic composition of the invention. These are fragments comprising at least 10 amino acids, preferably 20 amino acids, more preferably 30 amino acids, more preferably 40 amino acids or 50 amino acids, most preferably 100 amino acids, taken contiguously from the amino acid sequence of the protein. In addition, such immunogenic fragments are immunologically reactive with antibodies generated against the Bordetella proteins or with antibodies generated by infection of a mammalian host with Bordetella. Immunogenic fragments also include fragments that when administered at an effective dose, (either alone or as a hapten bound to a carrier), elicit a protective immune response against Bordetella infection, more preferably it is protective against Bordetella pertussis infection. Such an immunogenic fragment may include, for example, the protein lacking an N-terminal leader sequence, and/or a transmembrane domain and/or a C-terminal anchor domain. In one embodiment, an immunogenic fragment according to the invention comprises substantially all of the extracellular domain of a protein (e.g., at least 85%, preferably at least 90%, more preferably at least 95%, most preferably at least 97-99%, of the entire length of the extracellular domain of the protein).

Also included in immunogenic compositions of the invention are fusion proteins composed of Bordetella proteins, or immunogenic fragments of Bordetella proteins. Such fusion proteins may be made recombinantly and may comprise one portion of at least 2, 3, 4, 5 or 6 Bordetella proteins. Alternatively, a fusion protein may comprise multiple portions of at least 2, 3, 4 or 5 Bordetella proteins. These may combine different Bordetella proteins or immunogenic fragments thereof in the same protein. Alternatively, the invention also includes individual fusion proteins of Bordetella proteins or immunogenic fragments thereof, as a fusion protein with heterologous sequences such as a provider of T-cell epitopes or purification tags, for example: beta-galactosidase, glutathione-S-transferase, green fluorescent proteins (GFP), epitope tags such as FLAG, myc tag, poly histidine, or viral surface proteins such as influenza virus haemagglutinin, or bacterial proteins such as tetanus toxoid, diphtheria toxoid, CRM197. Extracellular component binding proteins are proteins that bind to host extracellular components. The term includes, but is not limited to adhesins. Examples of extracellular component binding proteins include filamentous hæmagglutinin adhesin (FHA), pertactin (PRN), finbrial-2 and fimbrail-3. FHA is a large, filamentous protein that serves as a dominant attachment factor for adherence to host ciliated epithelial cells of the respiratory tract, called respiratory epithelium. It is associated with biofilm formation and possesses at least four binding domains which can bind to different cell receptors on the epithelial cell surface.

FHA is a highly immunogenic, hairpin-shaped molecule which serves as the dominant attachment factor for Bordetella in animal model systems. Protein structure and immunological analyses suggest that the FHA proteins from B. pertussis and B. bronchiseptica are similar in their molecular mass, structure dimensions, and hemagglutination properties and have a common set of immunogenic epitopes.

FHA is synthesized as a 367-kDa precursor, FhaB, which undergoes extensive N- and C-terminal modifications to form the mature 220-kDa FHA protein. It is exported across the cytoplasmic membrane by a Sec signal peptide-dependent pathway. Its translocation and secretion across the outer membrane requires a specific accessory protein, FhaC. FhaC folds into a transmembrane β-barrel that facilitates secretion by serving as an FHA-specific pore in the outer membrane. FHA most probably crosses the outer membrane in an extended conformation and acquires its tertiary structure at the cell surface, following extensive N- and C-terminal proteolytic modifications. On translocation across the cytoplasmic membrane, the N terminus of FhaB undergoes cleavage of an additional 8 to 9 kDa at a site that corresponds to a Lep signal peptidase recognition sequence. This portion of the N terminus is predicted to be important for interacting with FhaC. Once at the cell surface, approximately 130 kDa of the C terminus of FhaB is proteolytically removed by a subtilisin-like autotransporter/protease, SphB1. FHA release depends on SphB1-mediated maturation. The C terminus of the FhaB precursor is predicted to serve as an intramolecular chaperone, preventing premature folding of the protein. Together, FHA and FhaC serve as prototypes for members of the two-partner secretion (TPS) system, which typically include secreted proteins with their cognate accessory proteins from several gram-negative bacteria. Although efficiently secreted via this process, a significant amount of FHA remains associated with the cell surface by an unknown mechanism.

FHA contains at least four separate binding domains that are involved in attachment. The Arg-Gly-Asp (RGD) triplet, situated in the middle of FHA and localized to one end of the proposed hairpin structure, stimulates adherence to monocytes/macrophages and possibly other leukocytes via the leukocyte response integrin/integrin-associated protein (LRI/IAP) complex and complement receptor type 3 (CR3). Specifically, the RGD motif of FHA has been implicated in binding to very late antigen 5 (VLA-5; an α₅β₁-integrin) of bronchial epithelial cells. Ligation of VLA-5 by the FHA RGD domain induces activation of NF-κB, which then causes the up-regulation of epithelial intercellular adhesion molecule 1 (ICAM-1). ICAM-1 up-regulation is involved in leukocyte accumulation and activation at the site of bacterial infection. FHA also possesses a carbohydrate recognition domain (CRD), which mediates attachment to ciliated respiratory epithelial cells as well as to macrophages in vitro. In addition, FHA displays a lectin-like activity for heparin and other sulfated carbohydrates, which can mediate adherence to nonciliated epithelial cell lines. This heparin-binding site is distinct from the CRD and RGD sites and is required for FHA-mediated hemagglutination. FHA is also required for biofilm formation in B. bronchiseptica.

Bordetella strains express a number of related surface-associated proteins belonging to the autotransporter secretion system. The autotransporter family includes functionally diverse proteins, such as proteases, adhesins, toxins, invasins, and lipases, that appear to direct their own export to the outer membrane. Autotransporters typically contain an N-terminal region called the passenger domain, which confers the effector functions, and a conserved C-terminal region called the β-barrel, which is required for the secretion of the passenger proteins across the membrane. The N-terminal signal sequence facilitates translocation of the preproprotein across the inner membrane via the Sec pathway. On cleavage of the N-terminal signal in the periplasm, the C terminus folds into a β-barrel in the outer membrane, forming an aqueous channel. The linker region between the N and C termini directs the translocation of the passenger through the β-barrel channel. On the surface, passenger domains may be cleaved from the translocation unit and remain noncovalently associated with the bacterial surface or may be released into the extracellular milieu following an autoproteolytic event (for example, when the passenger domain is a protease) or cleavage by an endogenous outer membrane protease.

Pertactin (PRN) is a member of the autotransporter family of Bordetella. Mature PRN is a 68-kDa protein in B. bronchiseptica, a 69-kDa protein in B. pertussis, and a 70-kDa protein in B. parapertussis (human). It has been proposed to play a role in attachment since all three PRN proteins contain an Arg-Gly-Asp (RGD) tripeptide motif as well as several proline-rich regions and leucine-rich repeats, motifs commonly present in molecules that form protein-protein interactions involved in eukaryotic cell binding. The B. pertussis, B. bronchiseptica, and B. parapertussis PRNs differ primarily in the number of proline-rich regions they contain. The X-ray crystal structure of B. pertussis PRN suggests that it contains 16-strand parallel β-helix with a V-shaped cross section and is the largest β-helix known to date. Deletion of the 3′ region of prnBp prevents surface exposure of the molecule.

Additional Bordetella proteins with autotransport ability include TcfA (originally classified as a tracheal colonization factor), BrkA, SphB1, and Vag8. All of these proteins show significant amino acid sequence similarity in their C termini and contain one or more RGD tripeptide motifs.

SphB1 has been characterized as a subtilisin-like Ser protease/lipoprotein that is essential for cleavage and C-terminal maturation of FHA. SphB1 is the first reported autotransporter whose passenger protein serves as a maturation factor for another protein secreted by the same organism. BrkA is expressed as a 103-kDa preproprotein that is processed to yield a 73-kDa α (passenger)-domain and a 30-kDa β-domain that facilitates transport by functioning dually as a secretion pore and an intramolecular chaperone that effects folding of the passenger concurrent with or following translocation across the outer membrane. Like PRN and SphB1, BrkA remains tightly associated with the bacterial surface. Vag8 is a 95-kDa outer membrane protein that is expressed in B. pertussis, B. bronchiseptica, and B. parapertussisₕᵤ. The B. pertussis and B. bronchiseptica Vag8 homologs are highly similar, and their C termini show significant homology to the C termini of PRN, BrkA, and TcfA, indicating that Vag8 functions as an autotransporter. TcfA is produced as a 90-kDa cell-associated precursor form that is processed to release a mature 60-kDa protein.

Fimbriae. Like many gram-negative pathogenic bacteria, Bordetella express filamentous, polymeric protein cell surface structures called fimbriae (FIM). The major fimbrial subunits that form the two predominant Bordetella fimbrial serotypes, Fim2 and Fim3 (AGG2 and AGG3), are encoded by unlinked chromosomal loci fim2 and fim3, respectively. A third unlinked locus, fimX, is expressed only at very low levels if at all, and recently a fourth fimbrial locus, fimN, was identified in B. bronchiseptica. B. bronchiseptica and B. parapertussis contain a fifth gene, fimA, located immediately upstream of the fimbrial biogenesis operon fimBCD and 3′ of fhaB, which is expressed and capable of encoding a fimbrial subunit type, FimA.

Adenylate cyclase (CyaA). All of the Bordetella species that infect mammals secrete CyaA, a bifunctional calmodulin-sensitive adenylate cyclase/hemolysin. CyaA is synthesized as a protoxin monomer of 1,706 amino acids. Its adenylate cyclase catalytic activity is located within the N-terminal 400 amino acids. The 1,300-amino-acid C-terminal domain mediates delivery of the catalytic domain into the cytoplasm of eukaryotic cells and possesses low but detectable hemolytic activity for sheep red blood cells. Amino acid sequence similarity between the C-terminal domain of CyaA, the hemolysins of E. coli (HlyA) and Actinobacillus pleuropneumoniae (HppA), and the leukotoxins of Pasteurella hemolytica (LktA) and Actinobacillus actinomycetemcomitans (AaLtA) places CyaA within a family of calcium-dependent, pore-forming cytotoxins known as RTX (repeats-in-toxin) toxins. Each of these toxins contains a tandem array of a nine amino acid repeat (LXGGXG(N/D)DX) thought to be involved in calcium binding. Before the CyaA protoxin can intoxicate host cells, it must be activated by the product of the cyaC gene, which is located adjacent to, and transcribed divergently from, the cyaABDE operon. CyaC activates the CyaA protoxin by catalyzing the palmitoylation of an internal lysine residue (Lys-983). The E. coli HlyA protoxin is also activated by fatty acyl group modification. Whereas E. coli hemoloysin is released in the extracellular medium, the majority of the Bordetella CyaA remains surface associated, with only a small portion being released in the supernatant. It was recently suggested that FHA may play a role in retaining CyaA toxin on the bacterial cell surface; B. pertussis mutants lacking FHA released significantly more CyaA into the medium, and CyaA toxin association with the bacterial surface could be restored by expressing FHA from a plasmid in trans. CyaA also inhibits biofilm formation in B. bronchiseptica, possibly via its interaction with FHA and subsequent interference with FHA function. The eukaryotic surface glycoprotein CD11b serves as the receptor for mature CyaA toxin.

Dermonecrotic toxin (DNT). Although initially misidentified as an endotoxin, DNT was one of the first B. pertussis virulence factors to be described. The DNTs of B. pertussis, B. bronchiseptica, and B. parapertussisₕᵤ are nearly identical (˜99% amino acid identity) cytoplasmic, single polypeptide chains of about 160 kDa. Bordetella DNT is a typical A-B toxin, composed of a 54-amino-acid N-terminal receptor-binding domain and a 300-amino-acid C-terminal enzymatic domain.

Lipopolysaccharides. Like endotoxins from other gram-negative bacteria, the LPS of Bordetella species are pyrogenic, mitogenic, and toxic and can activate and induce tumor necrosis factor production in macrophages. Bordetella LPS molecules differ in chemical structure from the well-known smooth-type LPS expressed by members of the family Enterobacteriaceae. Specifically, B. pertussis LPS lacks a repetitive O-antigenic structure and is therefore more similar to rough-type LPS. It resolves as two distinct bands (A and B) on silver-stained sodium dodecyl sulfate-polyacrylamide gels. The faster-migrating moiety, band B, consists of a lipid A molecule linked via a single ketodeoxyoctulosonic acid residue to a branched oligosaccharide core structure containing heptose, glucose, glucuronic acid, glucosamine, and galactosaminuronic acid (GalNAcA). The charged sugars, GalNAcA, glucuronic acid, and glucosamine, are not commonly found as core constituents in other LPS molecules. The slower-migrating moiety (band A) consists of band B plus a trisaccharide consisting of N-acetyl-N-methylfucosamine (FucNAcMe), 2,3-deoxy-di-N-acetylmannosaminuronic acid (2,3-diNAcManA), and N-acetylglucosamine (GlcNAc). B. bronchiseptica LPS is composed of band A and band B plus an O-antigen structure consisting of a single sugar polymer of 2,3-dideoxy-di-N-acetylgalactosaminuronic acid. B. parapertussisₕᵤ isolates contain LPS that lacks band A, has a truncated band B, and contains an O antigen that, like B. bronchiseptica, consists of 2,3-dideoxy-di-N-acetylgalactosaminuronic acid. B. parapertussisₒᵥ isolates lack O antigen and contain band A- and and B-like moieties that appear to be distinct from those of the other Bordetella species.

Type III secretion system (TTSS). A TTSS has been identified in Bordetella subspecies. TTSSs allow gram-negative bacteria to translocate effector proteins directly into the plasma membrane or cytoplasm of eukaryotic cells through a needle-like injection apparatus. These bacterial effector proteins then alter normal host cell-signaling cascades and other processes to promote the pathogenic strategies of the bacteria. Type III secretion has been identified in a variety of pathogens including those infecting humans, such as Yersinia, Shigella, Salmonella, and enteropathogenic E. coli, as well as the plant pathogens Pseudomonas syringae and Erwinia. The B. bronchiseptica TTSS contributes to persistent colonization of the trachea in both rat and mouse models of respiratory infection

Tracheal cytotoxin (TCT). TCT corresponds to a disaccharide-tetrapeptide monomer of peptidoglycan that is produced by all gram-negative bacteria as they break down and rebuild their cell wall during growth. Its structure is N-acetylglucosaminyl-1,6-anhydro-N-acetylmuramyl-(1)-alanyl-γ-(d)-glutamyl-mesodiaminopimelyl-(d)-alanine. While other bacteria, such as E. coli, recycle this peptidoglycan fragment by transporting it back into the cytoplasm via an integral cytoplasmic membrane protein called AmpG, Bordetella spp. release it into the environment due to the lack of a functional AmpG. As such, TCT is constitutively expressed and is independent of BvgAS control.

TCT causes mitochondrial bloating, disruption of tight junctions, and extrusion of ciliated cells, with little or no damage to nonciliated cells, in hamster tracheal ring cultures and a dose-dependent inhibition of DNA synthesis in HTE cells. TCT also causes loss of ciliated cells, cell blebbing, and mitochondrial damage, as is evident in human nasal epithelial biopsy specimens. TCT alone is necessary and sufficient to reproduce the specific ciliated-cell cytopathology characteristic of B. pertussis infection in explanted tracheal tissue. TCT-dependent increase in nitric oxide (NO) is proposed to mediate this severe destruction of ciliated cells. TCT triggers IL-1 α production in HTE cells, and both TCT and IL-1 α result in increased NO production when added to HTE cells. It is hypothesized that, in vivo, TCT stimulates IL-1 α production in nonciliated mucus-secreting cells, which positively controls the expression of inducible nitric oxide synthase, leading to high levels of NO production. NO then diffuses to neighboring ciliated cells, which are much more susceptible to its damaging effects. TCT also functions synergistically with Bordetella LPS to induce the production of NO within the airway epithelium.

Pertussis toxin (PT). PT is an ADP-ribosylating toxin synthesized and secreted exclusively by B. pertussis. It is an A-B toxin composed of six polypeptides, designated S1 to S5, which are encoded by the ptxA to ptxE genes, respectively. The S1 polypeptide comprises the A subunit of the toxin, while the pentameric B subunit consists of polypeptides S2, S3, S4, and S5 assembled in a 1:1:2:1 ratio. Each subunit is synthesized with an N-terminal signal sequence, suggesting that transport into the periplasmic space occurs via a general export pathway analogous to the sec system of E. coli. Secretion across the outer membrane requires a specialized transport apparatus composed of nine Ptl (for “pertussis toxin liberation”) proteins. The ptl locus bears extensive similarity to the prototype type IV secretion system involved in exporting single-stranded “T-DNA” encoded by the Agrobacterium tumefaciens virB operon, suggesting that both these systems function by a common mechanism to transport large protein complexes. Furthermore, there is evidence that only the fully assembled PT holotoxin is efficiently secreted.

The A component of PT, consisting of the enzymatically active S1 subunit, sits atop the B oligomer, a ringlike structure formed by the remaining S2 to S5 subunits. The subunits are held together by noncovalent interactions. The B oligomer binds to eukaryotic cell membranes and dramatically increases the efficiency with which the S1 subunit gains entry into host cells. It has been proposed that PT traverses the membrane directly without the need for endocytosis, since it does not require an acidic environment for entry into eukaryotic cells. Subsequent reports, however, have proposed that PT binds to cell surface receptors and undergoes endocytosis via a cytochalasin D-independent pathway. Early and late endosmes, as well as the Golgi apparatus, have been implicated in the PT trafficking process. Once within the host cell cytosol, the B oligomer intercalates into the cytoplasmic membrane and binds ATP, causing the release of the S1 subunit, which then becomes active on reduction of its disulfide bond.

The S1 subunit in its reduced form has been shown to catalyze the transfer of ADP-ribose from NAD to the a subunit of guanine nucleotide-binding proteins (G proteins) in eukaryotic cells. PT can bind ADP-ribosylate and thus inactivate G proteins such as Gᵢ, Gₜ (transducin), and Gₒ. When active, Gᵢ inhibits adenylyl cyclase and activates K⁺ channels, Gₜ activates cyclic GMP phosphodiesterase in specific photoreceptors, and Gₒ activates K⁺ channels, inactivates Ca²⁺ channels, and activates phospholipase C-β. Biological effects attributed to the disruption of these signaling pathways include histamine sensitization, enhancement of insulin secretion in response to regulatory signals, and both suppressive and stimulatory immunologic effects.

PT is a strong adjuvant in several immunologic systems in several animals and humans. This adjuvancy in the experimental-animal model is associated with enhancement of serum antibody responses to other antigens, increased cellular immune responses to various protein antigens, contribution to hyperacute experimental autoallergic encephalomyelitis, and increased anaphylactic sensitivity. Of these adjuvant activities demonstrated in animal model systems, only the enhancement of serum antibody responses to other vaccine antigens has been demonstrated to occur in vaccinated children.

Although, on the one hand, PT displays adjuvant properties, it has also been shown to inhibit chemotaxis, oxidative responses, and lysosomal enzyme release in neutrophils and macrophages. This phenotype has been confirmed using mouse and rat models, where PT was shown to inhibit chemotaxis and migration of neutrophils, monocytes/macrophages, and lymphocytes. Most recently, PT was shown to display an immunosuppressive activity, since mice infected with a PT mutant elicited much higher anti-Bordetella serum antibody titers than did mice infected with wild-type B. pertussis. PT has also been suggested to function as an adhesin involved in the adherence of B. pertussis to human macrophages and ciliated respiratory epithelial cells.

Other Antigens.

In one embodiment, one or more of the following proteins or products of specific genetic loci are included in an immunogenic composition of the invention.

Flagella. Bordetella flagella are peritrichous cell surface appendages required for motility.

Type IV pili. Bordetella contain polar pili usually with an N-methylated phenylalanine as the N-terminal residue. They may function in adherence, twitching motility, and DNA uptake.

Capsule. Bordetella capsules are a type II polysaccharide coat thought to be comprised of an N-acetylgalactosaminuronic acid Vi antigen-like polymer. They may function in protection against host defense mechanisms or survival in the environment.

Alcaligin. Bordetella contain alcaligin, a siderophore for complexing iron, which is internalized through outer membrane receptors (B. bronchiseptica encodes 16 such receptors while B. pertussis encodes 12). Iron uptake may be important for survival within mammalian hosts.

In one embodiment of the invention, an immunogenic composition comprises about 0.1 μg (or less than 0.1 μg) up to about 100 μg of one or more antigens described herein, and any amount in between, for example, about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 82 g, about 2.7 μg, about 2.8 μg, about 2.9 μg, about 3.0 μg, about 3.1 μg, about 3.2 μg, about 3.3 μg, about 3.4 μg, about 3.5 μg, about 3.6 μg, about 3.7 μg, about 3.8 μg, about 3.9 μg, about 4.0 μg, about 4.1 μg, about 4.2 μg, about 4.3 μg, about 4.4 μg, about 4.5 μg, about 4.6 μg, about 4.7 μg, about 4.8 μg, about 4.9 μg, about 5.0 μg, about 5.1 μg, about 5.2 μg, about 5.3 μg, about 5.4 μg, about 5.5 μg, about 5.6 μg, about 5.7 μg, about 5.8 μg, about 5.9 μg, about 6.0 μg, about 6.1 μg, about 6.2 μg, about 6.3 μg, about 6.4 μg, about 6.5 μg, about 6.6 μg, about 6.7 μg, about 6.8 μg, about 6.9 mg, about 7.0 mg, about 7.5 mg, about 8.0 mg, about 8.5 mg, about 9.0 mg, about 9.5 μg, about 10.0 μg, about 10.5 μg, about 11.0 μg, about 11.5 μg, about 12.0 μg, about 12.5 μg, about 13.0 μg, about 13.5 μg, about 14.0 μg, about 14.5 μg, about 15.0 μg, about 15.5 μg, about 16.0 μg, about 16.5 μg, about 17.0 μg, about 17.5 μg, about 18.0 μg, about 18.5 μg, about 19.0 μg, about 19.5 μg, about 20.0 μg, about 21.0 μg, about 22.0 μg, about 23.0 μg, about 24.0 μg, about 25.0 μg, about 26.0 μg, about 27.0 μg, about 28.0 μg, about 29.0 μg, about 30.0 μg, about 35.0 μg, about 40.0 μg, about 45.0 μg, about 50.0 μg, about 55.0 μg, about 60.0 μg, about 65.0 μg, about 70.0 μg, about 75.0 μg, about 80.0 μg, about 85.0 μg, about 90.0 μg, about 95.0 μg, or about 100.0 μg or more of one or more of each of the antigens (e.g., FHA, pertussis toxin and/or pertactin).

Preferred Combinations. A preferred combination of proteins in an immunogenic composition of the invention comprises pertussis toxin (Pt) and 1, 2, 3, 4 or 5 further antigens selected from the group consisting of filamentous hæmagglutinin adhesin (FHA), fimbriae, pertactin (PRN), Vag8, BrkA, SphB1, Tracheal colonization factor (TcfA), pertussis toxin (PT), adenylate cyclase (CyaA), Type III secretion, dermonectrotic toxin (DNT), Tracheal cytotoxin (TCT), and LPS (e.g., wlb locus, wbm locus, PagP).

A further preferred combination of proteins in an immunogenic composition of the invention comprises filamentous hæmagglutinin adhesin (FHA) and 1, 2, 3, 4 or 5 further antigens selected from the group consisting of fimbriae, pertactin (PRN), Vag8, BrkA, SphB1, Tracheal colonization factor (TcfA), pertussis toxin (PT), adenylate cyclase (CyaA), Type III secretion, dermonectrotic toxin (DNT), Tracheal cytotoxin (TCT), and LPS (e.g., wlb locus, wbm locus, PagP).

Another preferred combination of proteins in an immunogenic composition of the invention comprises pertactin (PRN) and 1, 2, 3, 4 or 5 further antigens selected from the group consisting of fimbriae, filamentous hæmagglutinin adhesin (FHA), Vag8, BrkA, SphB1, Tracheal colonization factor (TcfA), pertussis toxin (PT), adenylate cyclase (CyaA), Type III secretion, dermonectrotic toxin (DNT), Tracheal cytotoxin (TCT), and LPS (e.g., wlb locus, wbm locus, PagP).

A further preferred combination of proteins in an immunogenic composition of the invention comprises fimbriae and 1, 2, 3, 4 or 5 further antigens selected from the group consisting of filamentous hæmagglutinin adhesin (FHA), pertactin (PRN), Vag8, BrkA, SphB1, Tracheal colonization factor (TcfA), pertussis toxin (PT), adenylate cyclase (CyaA), Type III secretion, dermonectrotic toxin (DNT), Tracheal cytotoxin (TCT), and LPS (e.g., wlb locus, wbm locus, PagP).

A further preferred combination of proteins in an immunogenic composition of the invention comprises Vag8 and 1, 2, 3, 4 or 5 further antigens selected from the group consisting of filamentous hæmagglutinin adhesin (FHA), pertactin (PRN), fimbriae, BrkA, SphB1, Tracheal colonization factor (TcfA), pertussis toxin (PT), adenylate cyclase (CyaA), Type III secretion, dermonectrotic toxin (DNT), Tracheal cytotoxin (TCT), and LPS (e.g., wlb locus, wbm locus, PagP).

A further preferred combination of proteins in an immunogenic composition of the invention comprises BrkA and 1, 2, 3, 4 or 5 further antigens selected from the group consisting of filamentous hæmagglutinin adhesin (FHA), pertactin (PRN), fimbriae, Vag8, SphB1, Tracheal colonization factor (TcfA), pertussis toxin (PT), adenylate cyclase (CyaA), Type III secretion, dermonectrotic toxin (DNT), Tracheal cytotoxin (TCT), and LPS (e.g., wlb locus, wbm locus, PagP).

A further preferred combination of proteins in an immunogenic composition of the invention comprises SphB1 and 1, 2, 3, 4 or 5 further antigens selected from the group consisting of filamentous hæmagglutinin adhesin (FHA), pertactin (PRN), fimbriae, Vag8, BrkA, Tracheal colonization factor (TcfA), pertussis toxin (PT), adenylate cyclase (CyaA), Type III secretion, dermonectrotic toxin (DNT), Tracheal cytotoxin (TCT), and LPS (e.g., wlb locus, wbm locus, PagP).

A further preferred combination of proteins in an immunogenic composition of the invention comprises Tracheal colonization factor (TcfA) and 1, 2, 3, 4 or 5 further antigens selected from the group consisting of filamentous hæmagglutinin adhesin (FHA), pertactin (PRN), fimbriae, Vag8, BrkA, SphB1, pertussis toxin (PT), adenylate cyclase (CyaA), Type III secretion, dermonectrotic toxin (DNT), Tracheal cytotoxin (TCT), and LPS (e.g., wlb locus, wbm locus, PagP).

A further preferred combination of proteins in an immunogenic composition of the invention comprises adenylate cyclase (CyaA) and 1, 2, 3, 4 or 5 further antigens selected from the group consisting of filamentous hæmagglutinin adhesin (FHA), pertactin (PRN), fimbriae, Vag8, BrkA, SphB1, pertussis toxin (PT), Tracheal colonization factor (TcfA), Type III secretion, dermonectrotic toxin (DNT), Tracheal cytotoxin (TCT), and LPS (e.g., wlb locus, wbm locus, PagP).

A further preferred combination of proteins in an immunogenic composition of the invention comprises Type III secretion and 1, 2, 3, 4 or 5 further antigens selected from the group consisting of filamentous hæmagglutinin adhesin (FHA), pertactin (PRN), fimbriae, Vag8, BrkA, SphB1, pertussis toxin (PT), Tracheal colonization factor (TcfA), adenylate cyclase (CyaA), dermonectrotic toxin (DNT), Tracheal cytotoxin (TCT), and LPS (e.g., wlb locus, wbm locus, PagP).

A further preferred combination of proteins in an immunogenic composition of the invention comprises dermonectrotic toxin (DNT) and 1, 2, 3, 4 or 5 further antigens selected from the group consisting of filamentous hæmagglutinin adhesin (FHA), pertactin (PRN), fimbriae, Vag8, BrkA, SphB1, pertussis toxin (PT), Tracheal colonization factor (TcfA), adenylate cyclase (CyaA), Type III secretion, Tracheal cytotoxin (TCT), and LPS (e.g., wlb locus, wbm locus, PagP).

A further preferred combination of proteins in an immunogenic composition of the invention comprises Tracheal cytotoxin (TCT) and 1, 2, 3, 4 or 5 further antigens selected from the group consisting of filamentous hæmagglutinin adhesin (FHA), pertactin (PRN), fimbriae, Vag8, BrkA, SphB1, pertussis toxin (PT), Tracheal colonization factor (TcfA), adenylate cyclase (CyaA), Type III secretion, dermonectrotic toxin (DNT), and LPS (e.g., wlb locus, wbm locus, PagP).

A further preferred combination of proteins in an immunogenic composition of the invention comprises Bordetella LPS and 1, 2, 3, 4 or 5 further antigens selected from the group consisting of filamentous hæmagglutinin adhesin (FHA), pertactin (PRN), fimbriae, Vag8, BrkA, SphB1, pertussis toxin (PT), Tracheal colonization factor (TcfA), adenylate cyclase (CyaA), Type III secretion, dermonectrotic toxin (DNT), Tracheal cytotoxin (TCT).

As described in the Examples below, the invention provides that certain antigens produce a particularly effective immune response within the context of a mixture of antigens. Accordingly, an embodiment of the invention is an immunogenic composition comprising a Bordetella toxin (e.g., pertussis toxin) and a Bordetella extracellular binding protein (e.g., adhesion (e.g., FHA)), or a Bordetella toxin (e.g., pertussis toxin) and a Bordetella transporter protein (e.g., pertactin), or a Bordetella transporter protein (e.g., pertactin) and a Bordetella extracellular binding protein (e.g., adhesion (e.g., FHA)), or pertussis toxin and FHA, or pertactin and FHA, or pertactin and pertussis toxin. For each of these combinations, the proteins may be full length or fragments, having sequences at least 85%, 90%, 95%, 98% or 100% of the full length sequence (e.g., wild type or mutant sequence).

In the above and below combinations, the specified proteins may optionally be present in the immunogenic composition of the invention as a fragment or fusion protein.

A preferred immunogenic composition of the invention contains three protein components in a combination, for example, an extracellular component binding protein (FHA); a transporter protein (e.g., pertactin); and a regulator or virulence (e.g., pertussis toxin). For example, in one embodiment, the immunogenic composition contains a nanoemulsion and a combination of pertussis toxin, FHA and pertactin. Toxins may be chemically detoxified or genetically detoxified by introduction of point mutation(s). Toxins may also be present as a free protein or alternatively conjugated to a polysaccharide or other type of carbohydrate (e.g., an immunogenic carbohydrate moiety).

Polysaccharides and/or carbohydrate moieties may be of native size or alternatively may be sized, for instance by microfluidisation, ultrasonic irradiation or chemical cleavage. The invention also covers oligosaccharides extracted from Bordetella pertussis strains. Polysaccharides and/or carbohydrate moieties can be unconjugated or conjugated.

Conjugation of Polysaccharides and/or Carbohydrate Moieties

Problems associated with the use of polysaccharides and/or carbohydrate moieties in vaccination exist and are related to the fact that they are independently poor immunogens. Strategies, which have been designed to overcome this lack of immunogenicity, include the linking of the polysaccharide to large protein carriers, which provide bystander T-cell help. It is preferred that the polysaccharides utilized in the invention are linked to a protein carrier which provide bystander T-cell help. Examples of such carriers which may be conjugated to polysaccharide immunogens include the Diphtheria and Tetanus toxoids (DT, DT crm197 and TT respectively), Keyhole Limpet Haemocyanin (KLH), and the purified protein derivative of Tuberculin (PPD), Pseudomonas aeruginosa exoprotein A (rEPA), protein D from Haemophilus influenza, pneumolysin or fragments of any of the above. Fragments suitable for use include fragments encompassing T-helper epitopes. In particular protein D fragment will preferably contain the N-terminal ⅓ of the protein. Protein D is an IgD-binding protein from Haemophilus influenza (EP 0 594 610 B1) and is a potential immunogen.

In addition, Bordetella proteins may be used as carrier protein in the polysaccharide conjugates of the invention. The Bordetella proteins described below may be used as carrier protein; for example, filamentous hæmagglutinin adhesin (FHA), fimbriae, pertactin (PRN), Vag8, BrkA, SphB1, Tracheal colonization factor (TcfA), pertussis toxin (PT), adenylate cyclase (CyaA), Type III secretion, dermonectrotic toxin (DNT), Tracheal cytotoxin (TCT), or fragments thereof.

The polysaccharides may be linked to the carrier protein(s) by any known method (for example, by Likhite, U.S. Pat. No. 4,372,945 by Armor et al., U.S. Pat. No. 4,474,757, and Jennings et al., U.S. Pat. No. 4,356,170). Preferably, CDAP conjugation chemistry is carried out (see WO95/08348).

In CDAP, the cyanylating reagent 1-cyano-dimethylaminopyridinium tetrafluoroborate (CDAP) is preferably used for the synthesis of polysaccharide-protein conjugates. The cyanilation reaction can be performed under relatively mild conditions, which avoids hydrolysis of the alkaline sensitive polysaccharides. This synthesis allows direct coupling to a carrier protein.

The polysaccharide is solubilized in water or a saline solution. CDAP is dissolved in acetonitrile and added immediately to the polysaccharide solution. The CDAP reacts with the hydroxyl groups of the polysaccharide to form a cyanate ester. After the activation step, the carrier protein is added. Amino groups of lysine react with the activated polysaccharide to form an isourea covalent link. After the coupling reaction, a large excess of glycine is then added to quench residual activated functional groups. The product is then passed through a gel permeation column to remove unreacted carrier protein and residual reagents.

Conjugation preferably involves producing a direct linkage between the carrier protein and polysaccharide. Optionally a spacer (such as adipic dihydride (ADH)) may be introduced between the carrier protein and the polysaccharide.

Protection Against Bordetella Infection

In a preferred embodiment of the invention the immunogenic composition provides an effective immune response against more than one strain of Bordetella. More preferably, a protective immune response is generated against Bordetella pertussis.

In one embodiment, an effective immune response is defined as an immune response that gives significant protection in a rodent challenge model or bactericidal assay as described in the Examples. Significant protection in a rat challenge model, for instance that of example 1, is defined as an increase in the log₁₀ titer of Bordetella specific antibodies in comparison with control of at least 10%, 20%, 50%, 100% or 200%. Significant protection in a cotton rat challenge model, for instance that of Example 1, is defined as a decrease in the mean observed LogCFU of at least 10%, 20%, 50%, 70%, 80% or 90%.

Polynucleotide Vaccines. In a further aspect, the present invention relates to the use of a polynucleotides encoding a protein antigen described herein in the treatment, prevention or diagnosis of Bordetella infection. Such polynucleotides include isolated polynucleotides comprising a nucleotide sequence encoding a polypeptide which has at least 70% identity, preferably at least 80% identity, more preferably at least 90% identity, yet more preferably at least 95% identity, to the amino acid sequence of a wild type, full length antigen described herein.

Further polynucleotides that find utility in the present invention include isolated polynucleotides comprising a nucleotide sequence that has at least 70% identity, preferably at least 80% identity, more preferably at least 90% identity, yet more preferably at least 95% identity, to a nucleotide sequence encoding a protein of the invention over the entire coding region. In this regard, polynucleotides which have at least 97% identity are highly preferred, while those with at least 98-99% identity are more highly preferred, and those with at least 99% identity are most highly preferred. The polynucleotide can be inserted in a suitable plasmid or recombinant microorganism vector and used for expression (e.g., recombinant expression) and/or for immunization (see for example Wolff et. al., Science 247:1465-1468 (1990); Corr et. al., J. Exp. Med. 184:1555-1560 (1996); Doe et. al., Proc. Natl. Acad. Sci. 93:8578-8583 (1996)). The present invention also provides a nucleic acid encoding the aforementioned proteins of the present invention and their use in medicine. In a preferred embodiment isolated polynucleotides according to the invention may be single-stranded (coding or antisense) or double-stranded, and may be DNA (genomic, cDNA or synthetic) or RNA molecules. Additional coding or non-coding sequences may, but need not, be present within a polynucleotide of the present invention. The invention also contemplates the use of polynucleotides which are complementary to all the above described polynucleotides. The invention also provides for the use of a fragment (e.g., an immunogenic fragment) of a polynucleotide of the invention which when administered to a subject has the same immunogenic properties as a wild type, full length antigen of the invention.

Polynucleotides for use in the invention may be obtained, using standard cloning and screening techniques, from a cDNA library derived from mRNA in cells of human preneoplastic or tumor tissue (lung for example), (for example Sambrook et al., Molecular Cloning: A Laboratory Manual, 2.sup.nd Ed., Cold Spring harbor Laboratory Press, Cold Spring harbor, N.Y. (1989)). Polynucleotides of the invention can also be obtained from natural sources such as genomic DNA libraries or can be synthesized using well-known and commercially available techniques.

There are several methods available and well known to those skilled in the art to obtain full-length cDNAs, or extend short cDNAs, for example those based on the method of Rapid Amplification of cDNA ends (RACE) (see, for example, Frohman et al., PNAS USA 85, 8998-9002, 1988). Recent modifications of the technique, exemplified by the MARATHON technology (CLONTECH Laboratories Inc.) for example, have significantly simplified the search for longer cDNAs. In the MARATHON technology, cDNAs have been prepared from mRNA extracted from a chosen tissue and an ‘adaptor’ sequence ligated onto each end. Nucleic acid amplification (PCR) is then carried out to amplify the ‘missing’ 5′ end of the cDNA using a combination of gene specific and adaptor specific oligonucleotide primers. The PCR reaction is then repeated using ‘nested’ primers, that is, primers designed to anneal within the amplified product (typically an adaptor specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

Vectors comprising such DNA, hosts transformed thereby and the truncated or hybrid proteins themselves, expressed as described herein below all form part of the invention.

The expression system may also be a recombinant live microorganism, such as a virus or bacterium. The gene of interest can be inserted into the genome of a live recombinant virus or bacterium. Inoculation and in vivo infection with this live vector will lead to in vivo expression of the antigen and induction of immune responses.

Therefore, in certain embodiments, polynucleotides encoding immunogenic polypeptides for use according to the present invention are introduced into suitable mammalian host cells for expression using any of a number of known viral-based systems. In one illustrative embodiment, retroviruses provide a convenient and effective platform for gene delivery systems. A selected nucleotide sequence encoding a polypeptide for use in the present invention can be inserted into a vector and packaged in retroviral particles using techniques known in the art. The recombinant virus can then be isolated and delivered to a subject. A number of illustrative retroviral systems have been described (e.g., U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

In addition, a number of illustrative adenovirus-based systems have also been described. Unlike retroviruses which integrate into the host genome, adenoviruses persist extrachromosomally thus minimizing the risks associated with insertional mutagenesis (Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; and Rich et al. (1993) Human Gene Therapy 4:461-476).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional viral vectors useful for delivering the nucleic acid molecules encoding polypeptides for use in the present invention by gene transfer include those derived from the pox family of viruses, such as vaccinia virus and avian poxvirus. By way of example, vaccinia virus recombinants expressing the molecules of interest can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome.

The resulting TK.sup.(−) recombinant can be selected by culturing the cells in the presence of 5-bromodeoxyuridine and picking viral plaques resistant thereto.

A vaccinia-based infection/transfection system can be conveniently used to provide for inducible, transient expression or coexpression of one or more polypeptides described herein in host cells of an organism. In this particular system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide or polynucleotides of interest, driven by a T7 promoter. The polymerase expressed in the cytoplasm from the vaccinia virus recombinant transcribes the transfected DNA into RNA which is then translated into polypeptide by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al. Proc. Natl. Acad. Sci. USA (1986) 83:8122-8126.

Alternatively, avipoxviruses, such as the fowlpox and canarypox viruses, can also be used to deliver the coding sequences of interest. Recombinant avipox viruses, expressing immunogens from mammalian pathogens, are known to confer protective immunity when administered to non-avian species. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions for use in the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al. J. Biol. Chem. (1993) 268:6866-6869 and Wagner et al. Proc. Natl. Acad. Sci. USA (1992) 89:6099-6103, can also be used for gene delivery under the invention. Additional illustrative information on these and other known viral-based delivery systems can be found, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993.

The recombinant live microorganisms described above can be virulent, or attenuated in various ways in order to obtain live vaccines. Such live vaccines also form part of the invention.

In certain embodiments, a polynucleotide may be integrated into the genome of a target cell. This integration may be in the specific location and orientation via homologous recombination (gene replacement) or it may be integrated in a random, non-specific location (gene augmentation). In yet further embodiments, the polynucleotide may be stably maintained in the cell as a separate, episomal segment of DNA. Such polynucleotide segments or “episomes” encode sequences sufficient to permit maintenance and replication independent of or in synchronization with the host cell cycle. The manner in which the expression construct is delivered to a cell and where in the cell the polynucleotide remains is dependent on the type of expression construct employed.

In another embodiment of the invention, a polynucleotide is administered/delivered as “naked” DNA, for example as described in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

In still another embodiment, a composition of the present invention can be delivered via a particle bombardment approach, many of which have been described. In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

Nanoemulsions. In preferred embodiments, an immunogenic composition will be constructed with isolated antigens (e.g., isolated and /or recombinantly produced antigens) and an oil-in-water nanoemulsion.

Droplet Size. An immunogenic composition comprising nanoemulsion and a combination of Bordetella antigens of the invention comprises droplets having an average diameter size of less than about 1,000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 220 nm, less than about 210 nm, less than about 205 nm, less than about 200 nm, less than about 195 nm, less than about 190 nm, less than about 175 nm, less than about 150 nm, less than about 100 nm, greater than about 50 nm, greater than about 70 nm, greater than about 125 nm, or any combination thereof. In one embodiment, the droplets have an average diameter size greater than about 125 nm and less than or equal to about 600 nm. In a different embodiment, the droplets have an average diameter size greater than about 50 nm or greater than about 70 nm, and less than or equal to about 125 nm. In another embodiment, the droplets have an average diameter size between about 200 nm and about 400 nm

Aqueous Phase. The aqueous phase can comprise any type of aqueous phase including, but not limited to, water (e.g., H2O, distilled water, purified water, water for injection, de-ionized water, tap water) and solutions (e.g., phosphate buffered saline (PBS) solution). In certain embodiments, the aqueous phase comprises water at a pH of about 4 to 10, preferably about 6 to 8. The water can be deionized (hereinafter “DiH2O”). In some embodiments the aqueous phase comprises phosphate buffered saline (PBS). The aqueous phase may further be sterile and pyrogen free.

Organic Solvents. Organic solvents in the nanoemulsion of an immunogenic composition of the invention include, but are not limited to, C₁-C₁₂ alcohol, diol, triol, dialkyl phosphate, tri-alkyl phosphate, such as tri-n-butyl phosphate, semi-synthetic derivatives thereof, and combinations thereof. In one aspect of the invention, the organic solvent is an alcohol chosen from a nonpolar solvent, a polar solvent, a protic solvent, or an aprotic solvent.

Suitable organic solvents for the nanoemulsion of an immunogenic composition of the invention include, but are not limited to, ethanol, methanol, isopropyl alcohol, propanol, octanol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, polyethylene glycol, an organic phosphate based solvent, semi-synthetic derivatives thereof, and any combination thereof.

Oil Phase. The oil in the nanoemulsion of an immunogenic composition of the invention can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, CERAPHYLS, Decyl oleate, diisopropyl adipate, C12-15 alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the organic solvent, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives, or combinations thereof.

In one aspect of the invention, the volatile oil in the silicone component is different than the oil in the oil phase.

Surfactants. The surfactant in the nanoemulsion of an immunogenic composition of the invention can be a pharmaceutically acceptable ionic surfactant, a pharmaceutically acceptable nonionic surfactant, a pharmaceutically acceptable cationic surfactant, a pharmaceutically acceptable anionic surfactant, or a pharmaceutically acceptable zwitterionic surfactant.

Exemplary useful surfactants are described in Applied Surfactants: Principles and

Applications. Tharwat F. Tadros, Copyright 8 2005 WILEY-VCH Verlag GmbH & Co. KGaA, Weinheim ISBN: 3-527-30629-3), which is specifically incorporated by reference.

Further, the surfactant can be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof.

Surface active agents or surfactants, are amphipathic molecules that consist of a non-polar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thighlycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In additional embodiments, the surfactant is a polyoxyethylene fatty ether having a polyoxyethylene head group ranging from about 2 to about 100 groups, or an alkoxylated alcohol having the structure R5—(OCH2CH2)y—OH, wherein R5 is a branched or unbranched alkyl group having from about 6 to about 22 carbon atoms and y is between about 4 and about 100, and preferably, between about 10 and about 100. Preferably, the alkoxylated alcohol is the species wherein R5 is a lauryl group and y has an average value of 23.

In a different embodiment, the surfactant is an alkoxylated alcohol which is an ethoxylated derivative of lanolin alcohol. Preferably, the ethoxylated derivative of lanolin alcohol is laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis(imidazoyl carbonyl)), nonoxynol-9, Bis(polyethylene glycol bis[imidazoyl carbonyl]), BRIJ 35, BRIJ 56, BRIJ 72, BRIJ 76, BRIJ 92V, BRIJ 97, BRIJ 58P, CREMOPHOR, EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O—(N-heptylcarbamoyl)-alpha-D-glucopyranoside, Nonaethylene glycol monododecyl ether, N-Nonanoyl-N-methylglucamine, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, SPAN 20, SPAN 40, SPAN 60, SPAN 65, SPAN 80, SPAN 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, TRITON X-100, TRITON X-114, TRITON X-165, TRITON X-305, TRITON X-405, TRITON X-45, TRITON X-705-70, TWEEN 20, TWEEN 21, TWEEN 40, TWEEN 60, TWEEN 61, TWEEN 65, TWEEN 80, TWEEN 81, TWEEN 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, Poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable cationic surfactants include, but are not limited to, a quarternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride,

Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N,N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl) benzyl ammonium chloride, Alkyl demethyl benzyl ammonium chloride, Alkyl dimethyl 3,4-dichlorobenzyl ammonium chloride (100% O12), Alkyl dimethyl 3,4-dichlorobenzyl ammonium chloride (50% O14, 40% C12, 10% O16), Alkyl dimethyl 3,4-dichlorobenzyl ammonium chloride (55% C14, 23% C12, 20% O16), Alkyl dimethyl benzyl ammonium chloride, Alkyl dimethyl benzyl ammonium chloride (100% O14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25%

C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl dimethyl benzyl ammonium chloride, Alkyl didecyl dimethyl ammonium chloride, Alkyl dimethyl benzyl ammonium chloride, Alkyl dimethyl benzyl ammonium chloride (C12-16), Alkyl dimethyl benzyl ammonium chloride (C12-18), Alkyl dimethyl benzyl ammonium chloride, dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl-(ethylbenzyl) ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis(2-hydroxyethyl) octyl hydrogen ammonium chloride, Dodecyl dimethyl benzyl ammonium chloride, Dodecylcarbamoyl methyl dimethyl benzyl ammonium chloride, Heptadecyl hydroxyethylimidazolinium chloride, Hexahydro-1,3,5-tris(2-hydroxyethyl)-s-triazine, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Quaternary ammonium compounds, dicoco alkyldimethyl, chloride, Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with an particular cationic containing compound.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4, 1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, TRIZMA dodecyl sulfate, TWEEN 80, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, SigmaUltra, minimum 98% (TLC), CHAPS, for electrophoresis, minimum 98% (TLC), CHAPSO, minimum 98%, CHAPSO, SigmaUltra, CHAPSO, for electrophoresis, 3 -(Decyldimethylammonio)propanesulfonate inner salt, 3-Dodecyldimethyl-ammonio)propanesulfonate inner salt, SigmaUltra, 3-(Dodecyldimethylammonio)propanesulfonate inner salt, 3-(N,N-Dimethylmyristylammonio)propanesulfonate, 3-(N,N-Dimethylocatdecylammonio)propanesulfonate, 3-(N,N-Dimethyloctyl-ammonio)propanesulfonate inner salt, 3-(N,N-Dimethylpalmitylammonio)-propanesulfonate, semi-synthetic derivatives thereof, and combinations thereof.

In some embodiments, the nanoemulsion of an immunogenic composition of the invention comprises a cationic surfactant, which can be cetylpyridinium chloride. In other embodiments of the invention, the nanoemulsion of an immunogenic composition of the invention comprises a cationic surfactant, and the concentration of the cationic surfactant is less than about 5.0% and greater than about 0.001%. In yet another embodiment of the invention, the nanoemulsion of an immunogenic composition of the invention comprises a cationic surfactant, and the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, or less than about 0.10%. Further, the concentration of the cationic agent in the nanoemulsion of an immunogenic composition of the invention is greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, or greater than about 0.001%. In one embodiment, the concentration of the cationic agent in the nanoemulsion of an immunogenic composition of the invention is less than about 5.0% and greater than about 0.001%.

In another embodiment of the invention, the nanoemulsion of an immunogenic composition of the invention comprises at least one cationic surfactant and at least one non-cationic surfactant. The non-cationic surfactant is a nonionic surfactant, such as a polysorbate (Tween), such as polysorbate 80 or polysorbate 20. In one embodiment, the non-ionic surfactant is present in a concentration of about 0.01% to about 5.0%, or the non-ionic surfactant is present in a concentration of about 0.1% to about 3%. In yet another embodiment of the invention, the nanoemulsion of an immunogenic composition of the invention comprises a cationic surfactant present in a concentration of about 0.01% to about 2%, in combination with a nonionic surfactant.

In certain embodiments, the nanoemulsion of an immunogenic composition of the invention further comprises a cationic halogen containing compound. The present invention is not limited to a particular cationic halogen containing compound. A variety of cationic halogen containing compounds are contemplated including, but not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, and tetradecyltrimethylammonium halides. The nanoemulsion of an immunogenic composition of the invention is also not limited to a particular halide. A variety of halides are contemplated including, but not limited to, halide selected from the group consisting of chloride, fluoride, bromide, and iodide.

In still further embodiments, the nanoemulsion of an immunogenic composition of the invention further comprises a quaternary ammonium containing compound. The present invention is not limited to a particular quaternary ammonium containing compound. A variety of quaternary ammonium containing compounds are contemplated including, but not limited to, Alkyl dimethyl benzyl ammonium chloride, dialkyl dimethyl ammonium chloride, n-Alkyl dimethyl benzyl ammonium chloride, n-Alkyl dimethyl ethylbenzyl ammonium chloride, Dialkyl dimethyl ammonium chloride, and n-Alkyl dimethyl benzyl ammonium chloride.

In one embodiment, the nanoemulsion of an immunogenic composition of the invention comprises a cationic surfactant which is cetylpyridinium chloride (CPC). CPC may have a concentration in the nanoemulsion of an immunogenic composition of the invention of less than about 5.0% and greater than about 0.001%, or further, may have a concentration of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, less than about 0.10%, greater than about 0.001%, greater than about 0.002%, greater than about 0.003%, greater than about 0.004%, greater than about 0.005%, greater than about 0.006%, greater than about 0.007%, greater than about 0.008%, greater than about 0.009%, and greater than about 0.010%.

In a further embodiment, the nanoemulsion of an immunogenic composition of the invention comprises a non-ionic surfactant, such as a polysorbate surfactant, which may be polysorbate 80 or polysorbate 20, and may have a concentration of about 0.01% to about 5.0%, or about 0.1% to about 3% of polysorbate 80. The nanoemulsion of an immunogenic composition of the invention may further comprise at least one preservative. In another embodiment of the invention, the nanoemulsion of an immunogenic composition of the invention comprises a chelating agent.

Additional Ingredients. Additional compounds suitable for use in an immunogenic composition of the invention include but are not limited to one or more solvents, such as an organic phosphate-based solvent, bulking agents, coloring agents, pharmaceutically acceptable excipients, a preservative, pH adjuster, buffer, chelating agent, etc. The additional compounds can be admixed into a previously emulsified immunogenic composition comprising a nanoemulsion, or the additional compounds can be added to the original mixture to be emulsified. In certain of these embodiments, one or more additional compounds are admixed into an existing immunogenic composition immediately prior to its use.

Suitable preservatives in the immunogenic composition of the invention include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof. Other suitable preservatives include, but are not limited to, benzyl alcohol, chlorhexidine (bis(p-chlorophenyldiguanido) hexane), chlorphenesin (3-(-4-chloropheoxy)-propane-1,2-diol), Kathon CG (methyl and methylchloroisothiazolinone), parabens (methyl, ethyl, propyl, butyl hydrobenzoates), phenoxyethanol (2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens).

An immunogenic composition of the invention may further comprise at least one pH adjuster. Suitable pH adjusters in the immunogenic composition of the invention include, but are not limited to, diethyanolamine, lactic acid, monoethanolamine, triethylanolamine, sodium hydroxide, sodium phosphate, semi-synthetic derivatives thereof, and combinations thereof.

In addition, the immunogenic composition can comprise a chelating agent. In one embodiment of the invention, the chelating agent is present in an amount of about 0.0005% to about 1%. Examples of chelating agents include, but are not limited to, ethylenediamine, ethylenediaminetetraacetic acid (EDTA), phytic acid, polyphosphoric acid, citric acid, gluconic acid, acetic acid, lactic acid, and dimercaprol, and a preferred chelating agent is ethylenediaminetetraacetic acid.

The immunogenic compositions can comprise a buffering agent, such as a pharmaceutically acceptable buffering agent. Examples of buffering agents include, but are not limited to, 2-Amino-2-methyl-1,3-propanediol, ≧99.5% (NT), 2-Amino-2-methyl-1-propanol, ≧99.0% (GC), L-(+)-Tartaric acid, ≧99.5% (T), ACES, ≧99.5% (T), ADA, ≧99.0% (T), Acetic acid, ≧99.5% (GC/T), Acetic acid, for luminescence, ≧99.5% (GC/T), Ammonium acetate solution, for molecular biology, about 5 M in H2O, Ammonium acetate, for luminescence, ≧99.0% (calc. on dry substance, T), Ammonium bicarbonate, ≧99.5% (T), Ammonium citrate dibasic, ≧99.0% (T), Ammonium formate solution, 10 M in H2O, Ammonium formate, ≧99.0% (calc. based on dry substance, NT), Ammonium oxalate monohydrate, ≧99.5% (RT), Ammonium phosphate dibasic solution, 2.5 M in H2O, Ammonium phosphate dibasic, ≧99.0% (T), Ammonium phosphate monobasic solution, 2.5 M in H2O, Ammonium phosphate monobasic, ≧99.5% (T), Ammonium sodium phosphate dibasic tetrahydrate, ≧99.5% (NT), Ammonium sulfate solution, for molecular biology, 3.2 M in H2O, Ammonium tartrate dibasic solution, 2 M in H2O (colorless solution at 20.degree. C.), Ammonium tartrate dibasic, ≧99.5% (T), BES buffered saline, for molecular biology, 2.times. concentrate, BES, ≧99.5% (T), BES, for molecular biology, ≧99.5% (T), BICINE buffer Solution, for molecular biology, 1 M in H2O, BICINE, ≧99.5% (T), BIS-TRIS, ≧99.0% (NT), Bicarbonate buffer solution, >0.1 M Na2CO3, >0.2 M NaHCO3, Boric acid, ≧99.5% (T), Boric acid, for molecular biology, ≧99.5% (T), CAPS, ≧99.0% (TLC), CHES, ≧99.5% (T), Calcium acetate hydrate, ≧99.0% (calc. on dried material, KT), Calcium carbonate, precipitated, ≧99.0% (KT), Calcium citrate tribasic tetrahydrate, ≧98.0% (calc. on dry substance, KT), Citrate Concentrated Solution, for molecular biology, 1 M in H2O, Citric acid, anhydrous, ≧99.5% (T), Citric acid, for luminescence, anhydrous, ≧99.5% (T), Diethanolamine, ≧99.5% (GC), EPPS, ≧99.0% (T), Ethylenediaminetetraacetic acid disodium salt dihydrate, for molecular biology, ≧99.0% (T), Formic acid solution, 1.0 M in H2O, Gly-Gly-Gly, ≧99.0% (NT), Gly-Gly, ≧99.5% (NT), Glycine, ≧99.0% (NT), Glycine, for luminescence, ≧99.0% (NT), Glycine, for molecular biology, ≧99.0% (NT), HEPES buffered saline, for molecular biology, 2.times. concentrate, HEPES, ≧99.5% (T), HEPES, for molecular biology, ≧99.5% (T), Imidazole buffer Solution, 1 M in H2O, Imidazole, ≧99.5% (GC), Imidazole, for luminescence, ≧99.5% (GC), Imidazole, for molecular biology, ≧99.5% (GC), Lipoprotein Refolding Buffer, Lithium acetate dihydrate, >99.0% (NT), Lithium citrate tribasic tetrahydrate, ≧99.5% (NT), MES hydrate, ≧99.5% (T), MES monohydrate, for luminescence, ≧99.5% (T), MES solution, for molecular biology, 0.5 M in H2O, MOPS, ≧99.5% (T), MOPS, for luminescence, ≧99.5% (T), MOPS, for molecular biology, ≧99.5% (T), Magnesium acetate solution, for molecular biology, about 1 M in H2O, Magnesium acetate tetrahydrate, ≧99.0% (KT), Magnesium citrate tribasic nonahydrate, ≧98.0% (calc. based on dry substance, KT), Magnesium formate solution, 0.5 M in H2O, Magnesium phosphate dibasic trihydrate, ≧98.0% (KT), Neutralization solution for the in-situ hybridization for in-situ hybridization, for molecular biology, Oxalic acid dihydrate, ≧99.5% (RT), PIPES, ≧99.5% (T), PIPES, for molecular biology, ≧99.5% (T), Phosphate buffered saline, solution (autoclaved), Phosphate buffered saline, washing buffer for peroxidase conjugates in Western Blotting, 10 times. concentrate, piperazine, anhydrous, ≧99.0% (T), Potassium D-tartrate monobasic, ≧99.0% (T), Potassium acetate solution, for molecular biology, Potassium acetate solution, for molecular biology, 5 M in H2O, Potassium acetate solution, for molecular biology, about 1 M in H2O, Potassium acetate, ≧99.0% (NT), Potassium acetate, for luminescence, 99.0% (NT), Potassium acetate, for molecular biology, ≧99.0% (NT), Potassium bicarbonate, ≧99.5% (T), Potassium carbonate, anhydrous, ≧99.0% (T), Potassium chloride, ≧99.5% (AT), Potassium citrate monobasic, ≧99.0% (dried material, NT), Potassium citrate tribasic solution, 1 M in H2O, Potassium formate solution, 14 M in H2O, Potassium formate, ≧99.5% (NT), Potassium oxalate monohydrate, ≧99.0% (RT), Potassium phosphate dibasic, anhydrous, ≧99.0% (T), Potassium phosphate dibasic, for luminescence, anhydrous, ≧99.0% (T), Potassium phosphate dibasic, for molecular biology, anhydrous, ≧99.0% (T), Potassium phosphate monobasic, anhydrous, ≧99.5% (T), Potassium phosphate monobasic, for molecular biology, anhydrous, ≧99.5% (T), Potassium phosphate tribasic monohydrate, ≧95% (T), Potassium phthalate monobasic, ≧99.5% (T), Potassium sodium tartrate solution, 1.5 M in H2O, Potassium sodium tartrate tetrahydrate, ≧99.5% (NT), Potassium tetraborate tetrahydrate, ≧99.0% (T), Potassium tetraoxalate dihydrate, ≧99.5% (RT), Propionic acid solution, 1.0 M in H2O, STE buffer solution, for molecular biology, pH 7.8, STET buffer solution, for molecular biology, pH 8.0, Sodium 5,5-diethylbarbiturate, ≧99.5% (NT), Sodium acetate solution, for molecular biology, −3 M in H2O, Sodium acetate trihydrate, 99.5% (NT), Sodium acetate, anhydrous, ≧99.0% (NT), Sodium acetate, for luminescence, anhydrous, ≧99.0% (NT), Sodium acetate, for molecular biology, anhydrous, ≧99.0% (NT), Sodium bicarbonate, ≧99.5% (T), Sodium bitartrate monohydrate, ≧99.0% (T), Sodium carbonate decahydrate, ≧99.5% (T), Sodium carbonate, anhydrous, ≧99.5% (calc. on dry substance, T), Sodium citrate monobasic, anhydrous, ≧99.5% (T), Sodium citrate tribasic dihydrate, ≧99.0% (NT), Sodium citrate tribasic dihydrate, for luminescence, ≧99.0% (NT), Sodium citrate tribasic dihydrate, for molecular biology, ≧99.5% (NT), Sodium formate solution, 8 M in H2O, Sodium oxalate, ≧99.5% (RT), Sodium phosphate dibasic dihydrate, ≧99.0% (T), Sodium phosphate dibasic dihydrate, for luminescence, 99.0% (T), Sodium phosphate dibasic dihydrate, for molecular biology, ≧99.0% (T), Sodium phosphate dibasic dodecahydrate, ≧99.0% (T), Sodium phosphate dibasic solution, 0.5 M in H2O, Sodium phosphate dibasic, anhydrous, ≧99.5% (T), Sodium phosphate dibasic, for molecular biology, ≧99.5% (T), Sodium phosphate monobasic dihydrate, ≧99.0% (T), Sodium phosphate monobasic dihydrate, for molecular biology, ≧99.0% (T), Sodium phosphate monobasic monohydrate, for molecular biology, ≧99.5% (T), Sodium phosphate monobasic solution, 5 M in H2O, Sodium pyrophosphate dibasic, ≧99.0% (T), Sodium pyrophosphate tetrabasic decahydrate, ≧99.5% (T), Sodium tartrate dibasic dihydrate, ≧99.0% (NT), Sodium tartrate dibasic solution, 1.5 M in H2O (colorless solution at 20. degree. C.), Sodium tetraborate decahydrate, ≧99.5% (T), TAPS, ≧99.5% (T), TES, ≧99.5% (calc. based on dry substance, T), TM buffer solution, for molecular biology, pH 7.4,

TNT buffer solution, for molecular biology, pH 8.0, TRIS Glycine buffer solution, 10. times. concentrate, TRIS acetate-EDTA buffer solution, for molecular biology, TRIS buffered saline, 10. times. concentrate, TRIS glycine SDS buffer solution, for electrophoresis, 10. times. concentrate, TRIS phosphate-EDTA buffer solution, for molecular biology, concentrate, 10. times. concentrate, Tricine, ≧99.5% (NT), Triethanolamine, ≧99.5% (GC), Triethylamine, 99.5% (GC), Triethylammonium acetate buffer, volatile buffer, −1.0 M in H2O, Triethylammonium phosphate solution, volatile buffer, about 1.0 M in H2O, Trimethylammonium acetate solution, volatile buffer, about 1.0 M in H2O, Trimethylammonium phosphate solution, volatile buffer, about 1 M in H2O, Tris-EDTA buffer solution, for molecular biology, concentrate, 100. times. concentrate, Tris-EDTA buffer solution, for molecular biology, pH 7.4, Tris-EDTA buffer solution, for molecular biology, pH 8.0, TRIZMA acetate, ≧99.0% (NT), TRIZMA base, ≧99.8% (T), TRIZMA base, ≧99.8% (T), TRIZMA base, for luminescence, ≧99.8% (T), TRIZMA base, for molecular biology, ≧99.8% (T), TRIZMA carbonate, ≧98.5% (T), TRIZMA hydrochloride buffer solution, for molecular biology, pH 7.2, TRIZMA hydrochloride buffer solution, for molecular biology, pH 7.4, TRIZMA hydrochloride buffer solution, for molecular biology, pH 7.6, TRIZMA hydrochloride buffer solution, for molecular biology, pH 8.0, TRIZMA hydrochloride, ≧99.0% (AT), TRIZMA hydrochloride, for luminescence, ≧99.0% (AT), TRIZMA hydrochloride, for molecular biology, ≧99.0% (AT), and TRIZMA maleate, ≧99.5% (NT).

The immunogenic composition can comprise one or more emulsifying agents to aid in the formation of emulsions. Emulsifying agents include compounds that aggregate at the oil/water interface to form a kind of continuous membrane that prevents direct contact between two adjacent droplets. Certain embodiments of the present invention feature immunogenic compositions that may readily be diluted with water or another aqueous phase to a desired concentration without impairing their desired properties.

Immune Modulators. As noted above, immunogenic compositions of the invention can further comprise one or more immune modulators. Examples of immune modulators include, but are not limited to, chitosan and glucan. An immune modulator can be present in the immunogenic composition at any pharmaceutically acceptable amount including, but not limited to, from about 0.001% up to about 10%, and any amount in between, such as about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10%.

Pharmaceutical Compositions. An immunogenic composition of the invention may be formulated into pharmaceutical compositions that comprise the immunogenic composition in a therapeutically effective amount and suitable, pharmaceutically-acceptable excipients for pharmaceutically acceptable delivery. Such excipients are well known in the art.

By the phrase “therapeutically effective amount” it is meant any amount of the immunogenic composition that is effective in preventing, treating or ameliorating a disease caused by a Bordetella (e.g., B. pertussis). By “protective immune response” it is meant that the immune response is associated with prevention, treating, or amelioration of a disease. Complete prevention is not required, though is encompassed by the present invention. The immune response can be evaluated using the methods discussed herein or by any method known by a person of skill in the art.

Intranasal administration includes administration via the nose, either with or without concomitant inhalation during administration. Such administration is typically through contact by the composition comprising the immunogenic composition with the nasal mucosa, nasal turbinates or sinus cavity. Administration by inhalation comprises intranasal administration, or may include oral inhalation. Such administration may also include contact with the oral mucosa, bronchial mucosa, and other epithelia.

Exemplary dosage forms for pharmaceutical administration are described herein. Examples include but are not limited to liquids, ointments, creams, emulsions, lotions, gels, bioadhesive gels, sprays, aerosols, pastes, foams, sunscreens, capsules, microcapsules, suspensions, pessary, powder, semi-solid dosage form, etc.

A pharmaceutical immunogenic composition may be formulated for immediate release, sustained release, controlled release, delayed release, or any combinations thereof, into the epidermis or dermis. In some embodiments, the formulations may comprise a penetration-enhancing agent. Suitable penetration-enhancing agents include, but are not limited to, alcohols such as ethanol, triglycerides and aloe compositions. The amount of the penetration-enhancing agent may comprise from about 0.5% to about 40% by weight of the formulation.

The immunogenic compositions of the invention can be applied and/or delivered utilizing electrophoretic delivery/electrophoresis. Further, the composition may be a transdermal delivery system such as a patch or administered by a pressurized or pneumatic device (i.e., “gene gun”). Such methods, which comprise applying an electrical current, are well known in the art.

The immunogenic compositions for administration may be applied in a single administration or in multiple administrations.

If applied topically, the immunogenic compositions may be occluded or semi-occluded. Occlusion or semi-occlusion may be performed by overlaying a bandage, polyoleofin film, article of clothing, impermeable barrier, or semi-impermeable barrier to the topical preparation.

An exemplary nanoemulsion according to the invention is designated “W805EC.” The composition of W805EC is shown in Table 1. The mean droplet size for the W805EC adjuvant is about 400 nm. All of the components of the nanoemulsion are included on the FDA inactive ingredient list for Approved Drug Products.

TABLE 1
W805EC nanoemulsion formulation.
W₈₀5EC Formulation

| | W₈₀5EC-Adjuvant |
|---|---|
| Function | Mean Droplet Size ≈ 400 nm |
| Aqueous Diluent | Purified Water, USP |
| Hydrophobic Oil (Core) | Soybean Oil, USP (super refined) |
| Organic Solvent | Dehydrated Alcohol, USP (anhydrous |
| | ethanol) |
| Surfactant | Polysorbate 80, NF |
| Emulsifying Agent | Cetylpyridinium Chloride, USP |
| Preservative | |

In one embodiment, nanoemulsions are formed by emulsification of an oil, purified water, nonionic detergent, organic solvent and surfactant, such as a cationic surfactant. An exemplary specific nanoemulsion of an immunogenic composition of the invention is designated as “60% W805EC”. The 60% W805EC-formulation is composed of the ingredients shown in Table 2: purified water, USP; soybean oil USP; Dehydrated Alcohol, USP [anhydrous ethanol]; Polysorbate 80, NF and cetylpyridinium chloride, USP(CPCAII components of this exemplary nanoemulsion are included on the FDA list of approved inactive ingredients for Approved Drug Products.

TABLE 2
60% W805EC-formulation.
Composition of 60% W₈₀5EC-Adjuvant (w/w %)

| Ingredients | 60% W₈₀5EC |
|---|---|
| Purified Water, USP | 54.10% |
| Soybean Oil, USP | 37.67% |
| Dehydrated Alcohol, USP | 4.04% |
| (anhydrous ethanol) | |
| Polysorbate 80, NF | 3.55% |
| Cetylpyridinium Chloride, USP | 0.64% |

Methods of Manufacture. A nanoemulsion of an immunogenic composition of the invention can be formed using classic emulsion forming techniques. See e.g., U.S. 2004/0043041. In an exemplary method, the oil is mixed with the aqueous phase under relatively high shear forces (e.g., using high hydraulic and mechanical forces) to obtain a nanoemulsion comprising oil droplets having an average diameter of less than about 1000 nm. Some embodiments of the invention employ a nanoemulsion having an oil phase comprising an alcohol such as ethanol. The oil and aqueous phases can be blended using any apparatus capable of producing shear forces sufficient to form an emulsion, such as French Presses or high shear mixers (e.g., FDA approved high shear mixers are available, for example, from Admix, Inc., Manchester, N.H.). Methods of producing such emulsions are described in U.S. Pat. Nos. 5,103,497 and 4,895,452, herein incorporated by reference in their entireties.

In an exemplary embodiment, a nanoemulsion of an immunogenic composition used in the methods of the invention comprise droplets of an oily discontinuous phase dispersed in an aqueous continuous phase, such as water or PBS. The nanoemulsions of the invention are stable, and do not deteriorate even after long storage periods. Certain nanoemulsions of the invention are non-toxic and safe when swallowed, inhaled, or contacted to the skin of a subject.

A nanoemulsion of an immunogenic composition of the invention can be produced in large quantities and be stable for many months at a broad range of temperatures. The nanoemulsion can have textures ranging from that of a semi-solid cream to that of a thin lotion, to that of a liquid and can be applied topically by any pharmaceutically acceptable method as stated above, e.g., by hand, or nasal drops/spray.

As stated above, at least a portion of the emulsion may be in the form of lipid structures including, but not limited to, unilamellar, multilamellar, and paucliamellar lipid vesicles, micelles, and lamellar phases.

The present invention contemplates that many variations of the described nanoemulsions will be useful in immunogenic compositions and methods of the present invention. To determine if a candidate nanoemulsion is suitable for use with the present invention, three criteria are analyzed. Using the methods and standards described herein, candidate emulsions can be easily tested to determine if they are suitable. First, the desired ingredients are prepared using the methods described herein, to determine if a nanoemulsion can be formed. If a nanoemulsion cannot be formed, the candidate is rejected. Second, the candidate nanoemulsion should form a stable emulsion. A nanoemulsion is stable if it remains in emulsion form for a sufficient period to allow its intended use. For example, for nanoemulsions that are to be stored, shipped, etc., it may be desired that the nanoemulsion remain in emulsion form for months to years. Typical nanoemulsions that are relatively unstable, will lose their form within a day. Third, the candidate nanoemulsion should have efficacy for its intended use. For example, the emulsions of the invention should maintain (e.g., not decrease or diminish) and/or enhance the immunogenicity of antigen (e.g., B. pertussis antigens), or induce a protective immune response to a detectable level (e.g., when used in combination with one or a plurality of antigens (e.g., B. pertussis antigens). The nanoemulsion of the invention can be provided in many different types of containers and delivery systems. For example, in some embodiments of the invention, the nanoemulsions are provided in a cream or other solid or semi-solid form. The nanoemulsions of the invention may be incorporated into hydrogel formulations.

The nanoemulsions can be delivered (e.g., to a subject or customers) in any suitable container. Suitable containers can be used that provide one or more single use or multi-use dosages of the nanoemulsion for the desired application. In some embodiments of the invention, the nanoemulsions are provided in a suspension or liquid form. Such nanoemulsions can be delivered in any suitable container including spray bottles and any suitable pressurized spray device. Such spray bottles may be suitable for delivering the nanoemulsions intranasally or via inhalation. These nanoemulsion-containing containers can further be packaged with instructions for use to form kits.

An exemplary method for manufacturing an immunogenic composition according to the invention for the treatment or prevention of Bordetella (e.g., B. pertussis) infection in humans comprises: (1) synthesizing in an eukaryotic host, one or more Bordetella antigens; and/or (2) synthesizing in an eukaryotic host, one or more Bordetella antigens, wherein the synthesizing is performed utilizing recombinant DNA genetics vectors and constructs. The one or more Bordetella antigens can then be isolated from the eukaryotic host, followed by formulating the one or more Bordetella antigens with an oil in water nanoemulsion. The eukaryotic host can be, for example, a mammalian cell, a yeast cell, or an insect cell.

Vaccines. In a preferred embodiment, the immunogenic composition of the invention is utilized as, or mixed with a pharmaceutically acceptable excipient (e.g., an adjuvant) to form, a vaccine. In a further preferred embodiment, an immunogenic composition (e.g., vaccine) of the invention contains an oil in water nanoemulsion and one or a plurality of Bordetella (e.g., B. pertussis) antigens and does not include an adjuvant.

In another embodiment, the vaccines of the present invention are adjuvanted. Suitable adjuvants include an aluminum salt such as aluminum hydroxide gel (alum) or aluminum phosphate, but may also be a salt of calcium, magnesium, iron or zinc, or may be an insoluble suspension of acylated tyrosine, or acylated sugars, cationically or anionically derivatized polysaccharides, or polyphosphazenes.

In one embodiment, the adjuvant is selected to be a preferential inducer of either a TH1 or a TH2 type of response. High levels of Th1-type cytokines tend to favor the induction of cell mediated immune responses to a given antigen, while high levels of Th2-type cytokines tend to favor the induction of humoral immune responses to the antigen. It is important to remember that the distinction of Th1 and Th2-type immune response is not absolute. In reality an individual will support an immune response which is described as being predominantly Th1 or predominantly Th2. However, it is often convenient to consider the families of cytokines in terms of that described in murine CD4 +ve T cell clones by Mosmann and Coffman (Mosmann, T. R. and Coffman, R. L. (1989) TH1 and TH2 cells: different patterns of lymphokine secretion lead to different functional properties. Annual Review of Immunology, 7, p 145-173). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of I1-4, IL-5, IL-6, IL-10. Suitable adjuvant systems which promote a predominantly Th1 response include: Monophosphoryl lipid A or a derivative thereof, particularly 3-de-O-acylated monophosphoryl lipid A (3D-MPL) (for its preparation see GB 2220211 A); and a combination of monophosphoryl lipid A, preferably 3-de-O-acylated monophosphoryl lipid A, together with either an aluminum salt (for instance aluminum phosphate or aluminum hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen (See, Thoelen et al. Vaccine (1998) 16:708-14; EP 689454-B1).

An enhanced system involves the combination of a monophosphoryl lipid A and a saponin derivative, particularly the combination of QS21 and 3D-MPL as disclosed in WO 94/00153, or a less reactogenic composition where the QS21 is quenched with cholesterol as disclosed in WO 96/33739. A particularly potent adjuvant formulation involving QS21, 3D-MPL and tocopherol in an oil in water emulsion is described in WO 95/17210, and is a preferred formulation. Preferably the vaccine additionally comprises a saponin, more preferably QS21. The formulation may also comprise an oil in water emulsion and tocopherol (WO 95/17210). The present invention also provides a method for producing a vaccine formulation comprising mixing an antigen(s) of the present invention together with a pharmaceutically acceptable excipient, such as 3D-MPL. Unmethylated CpG containing oligonucleotides (WO 96/02555) are also preferential inducers of a TH1 response and are suitable for use in the present invention.

In one embodiment, immunogenic compositions of the invention form a liposome structure. Compositions where the sterol/immunologically active saponin fraction forms an ISCOM structure also form an aspect of the invention.

The ratio of QS21:sterol will typically be in the order of 1:100 to 1:1 weight to weight. Preferably excess sterol is present, the ratio of QS21:sterol being at least 1:2 w/w. Typically for human administration QS21 and sterol will be present in a vaccine in the range of about 1 μg to about 100 μg, preferably about 10 μg to about 50 μg per dose.

The liposomes preferably contain a neutral lipid, for example phosphatidylcholine, which is preferably non-crystalline at room temperature, for example egg yolk phosphatidylcholine, dioleoyl phosphatidylcholine or dilauryl phosphatidylcholine. The liposomes may also contain a charged lipid which increases the stability of the liposome-QS21 structure for liposomes composed of saturated lipids. In these cases the amount of charged lipid is preferably 1-20% w/w, most preferably 5-10%. The ratio of sterol to phospholipid is 1-50% (mol/mol), most preferably 20-25%.

In another embodiment, compositions of the invention contain MPL (3-deacylated mono-phosphoryl lipid A, also known as 3D-MPL). 3D-MPL is known from GB 2 220 211 (Ribi) as a mixture of 3 types of De-O-acylated monophosphoryl lipid A with 4, 5 or 6 acylated chains and is manufactured by Ribi Immunochem, Montana. A preferred form is disclosed in International Patent Application 92/116556.

In other embodiments, compositions of the invention are those wherein liposomes are initially prepared without MPL, and MPL is then added, preferably as 100 nm particles. The MPL is therefore not contained within the vesicle membrane (known as MPL out). Compositions where the MPL is contained within the vesicle membrane (known as MPL in) also form an aspect of the invention. The antigen can be contained within the vesicle membrane or contained outside the vesicle membrane. Preferably soluble antigens are outside and hydrophobic or lipidated antigens are either contained inside or outside the membrane.

A vaccine preparation of the present invention may be used to protect or treat a mammal susceptible to infection, by means of administering the vaccine via systemic or mucosal route. These administrations may include injection via the intramuscular, intraperitoneal, intradermal or subcutaneous routes; or via mucosal administration to the oral/alimentary, respiratory, genitourinary tracts. In a preferred embodiment, the present invention provides intranasal administration of vaccines for the treatment of pertussis (e.g., nasopharyngeal carriage of B. pertussis is effectively prevented, thus attenuating infection at its earliest stage). Thus, in one embodiment, an immunogenic composition (e.g., vaccine) of the invention is administered mucosally (e.g., intranasally) to a host subject thereby reducing and/or eliminating colonization and/or carriage of B. pertussis in the nasopharynx of the host. Although a vaccine of the invention may be administered as a single dose, components thereof may also be co-administered together at the same time or at different times (for instance B. pertussis LPS could be administered separately, at the same time or 1-2 weeks after the administration of any B. pertussis antigen component of the vaccine (e.g., FHA, pertussis toxin and/or pertactin) for optimal coordination of the immune responses with respect to each other). For co-administration, the optional Th1 adjuvant may be present in any or all of the different administrations, however it is preferred if it is present in combination with a protein component of the vaccine. In addition to a single route of administration, 2 different routes of administration may be used. For example, polysaccharides may be administered IM (or ID) and proteins may be administered IN. In addition, the vaccines of the invention may be administered IM for priming doses and IN for booster doses, or, may be administered IN for priming doses and IM for booster doses.

The amount of conjugate antigen in each vaccine dose is selected as an amount which induces an immunoprotective response without significant, adverse side effects in typical vaccines. Such amount will vary depending upon which specific immunogen is employed and how it is presented. Generally, it is expected that each dose will comprise 0.1-100 μg of polysaccharide, preferably 0.1-50 μg for polysaccharide conjugates, preferably 0.1-10 μg, more preferably 1-10 μg, of which 1 to 5 μg is a more preferable range.

The content of protein antigens in the vaccine will typically be in the range 1-100 μg, preferably 5-50 μg, most typically in the range 5-25 μg. Following an initial vaccination, subjects may receive one or several booster immunizations adequately spaced.

Vaccine preparation is generally described in Vaccine Design (“The subunit and adjuvant approach” (eds Powell M. F. & Newman M. J.) (1995) Plenum Press New York). Encapsulation within liposomes is described by Fullerton, U.S. Pat. No. 4,235,877.

In some embodiments, the vaccines of the present invention are stored in solution or lyophilized. If lyophilized, preferably the solution is lyophilized in the presence of a sugar such as sucrose, trehalose or lactose. It is still further preferable that they are lyophilized and extemporaneously reconstituted prior to use. Lyophilizing may result in a more stable composition (vaccine) and may possibly lead to higher antibody titers in the presence of 3D-MPL and in the absence of an aluminum based adjuvant.

Antibodies and Passive Immunization

Another aspect of the invention is a method of preparing an immune globulin for use in prevention or treatment of Bordetella (B. pertussis) infection comprising the steps of immunizing a recipient with a vaccine of the invention and isolating immune globulin from the recipient. An immune globulin prepared by this method is a further aspect of the invention. A pharmaceutical composition comprising the immune globulin of the invention and a pharmaceutically acceptable carrier is a further aspect of the invention which could be used in the manufacture of a medicament for the treatment or prevention of Bordetella (B. pertussis) disease. A method for treatment or prevention of Bordetella (B. pertussis) infection comprising a step of administering to a patient an effective amount of the pharmaceutical preparation of the invention is a further aspect of the invention.

Inocula for polyclonal antibody production are typically prepared by dispersing the antigenic composition in a physiologically tolerable diluent such as saline or other adjuvants suitable for human use to form an aqueous composition. An immunostimulatory amount of inoculum is administered to a mammal and the inoculated mammal is then maintained for a time sufficient for the antigenic composition to induce protective antibodies.

The antibodies can be isolated to the extent desired by well-known techniques such as affinity chromatography (Harlow and Lane Antibodies; a laboratory manual 1988).

Antibodies can include antiserum preparations from a variety of commonly used animals e.g. goats, primates, donkeys, swine, horses, guinea pigs, rats or man. The animals are bled and serum recovered.

An immune globulin produced in accordance with the present invention can include whole antibodies, antibody fragments or subfragments. Antibodies can be whole immunoglobulins of any class e.g. IgG, IgM, IgA, IgD or IgE, chimeric antibodies or hybrid antibodies with dual specificity to two or more antigens of the invention. They may also be fragments e.g. F(ab′)2, Fab′, Fab, Fv and the like including hybrid fragments. An immune globulin also includes natural, synthetic or genetically engineered proteins that act like an antibody by binding to specific antigens to form a complex.

A vaccine of the present invention can be administered to a recipient who then acts as a source of immune globulin, produced in response to challenge from the specific vaccine. A subject thus treated would donate plasma from which hyperimmune globulin would be obtained via conventional plasma fractionation methodology. The hyperimmune globulin would be administered to another subject in order to impart resistance against or treat Bordetella (B. pertussis) infection. Hyperimmune globulins of the invention are particularly useful for treatment or prevention of Bordetella (B. pertussis) disease in infants, immune compromised individuals or where treatment is required and there is no time for the individual to produce antibodies in response to vaccination.

An additional aspect of the invention is a pharmaceutical composition comprising two of more monoclonal antibodies (or fragments thereof; preferably human or humanized) reactive against at least two constituents of the immunogenic composition of the invention, which could be used to treat or prevent infection by Bordetella (B. pertussis).

Such pharmaceutical compositions comprise monoclonal antibodies that can be whole immunoglobulins of any class e.g. IgG, IgM, IgA, IgD or IgE, chimeric antibodies or hybrid antibodies with specificity to two or more antigens of the invention. They may also be fragments e.g. F(ab′)2, Fab′, Fab, Fv and the like including hybrid fragments.

Methods of making monoclonal antibodies are well known in the art and can include the fusion of splenocytes with myeloma cells (Kohler and Milstein 1975 Nature 256; 495; Antibodies—a laboratory manual Harlow and Lane 1988). Alternatively, monoclonal Fv fragments can be obtained by screening a suitable phage display library (Vaughan T J et al 1998 Nature Biotechnology 16; 535). Monoclonal antibodies may be humanized or part humanized by known methods.

Methods of Treatment

Immunogenic compositions of the present invention described herein may be used to protect or treat a mammal (e.g., a human) susceptible to infection, by means of administering the immunogenic composition via systemic or mucosal route. These administrations may include injection via the intramuscular, intraperitoneal, intradermal or subcutaneous routes; or via mucosal administration to the oral/alimentary, respiratory, genitourinary tracts.

The invention also encompasses method of treatment of Bordetella (B. pertussis) infection. An immunogenic composition or vaccine of the invention is particularly advantageous to use in cases of an outbreak of pertussis in a community.

As described herein, the invention provides methods of preventing and/or treating infection and/or disease caused by a species of Bordetella (e.g., B. pertussis (e.g., whooping cough)) comprising administering an effective amount of an immunogenic composition of the invention to a subject. For example, the invention provides the use of an immunogenic composition of the invention for the manufacture of a medicament (e.g., a vaccine) for the treatment of Bordetella (e.g., B. pertussis) infection (e.g., whooping cough). The invention also provides an immunogenic composition (e.g., any one of the immunogenic compositions of the invention) for use in the treatment of Bordetella (e.g., B. pertussis) infection. For example, in some embodiments, methods of treating subjects protects the subject against B. pertussis colonization (e.g., prevents a subject administered the immunogenic composition against infection and disease caused by B. pertussis and/or eliminates carriage of B. pertussis in subjects administered the immunogenic composition (e.g., thereby providing herd immunity and/or eliminating B. pertussis from a population of subjects)). While an understanding of a mechanism of action is not needed to practice the present invention, and while the present invention is not limited to any particular mechanism of action, in one embodiment, administration of an immunogenic composition of the invention confers systemic and mucosal immunity and protects against colonization and transmission of B. pertussis (e.g., induces a Th17 type immune response in the vaccinated subject that in turn blocks colonization, carriage and/or transmission of B. pertussis within the subject and/or a population of subjects in which the subject resides). Thus, in a preferred embodiment, intranasal administration of an immunogenic composition of the invention reduces and/or eliminates carriage of B. pertussis (e.g., in a subject administered the immunogenic composition and/or to others in the population not administered the composition (e.g., herd immunity). The invention is not limited by the type of subject administered an immunogenic composition of the invention. Indeed, any subject that can be administered an effective amount of an immunogenic composition of the invention (e.g., to induce an immune response specific to B. pertussis in the subject) is contemplated to benefits from the immunogenic compositions of the invention. In one embodiment, the subject is an adult (e.g., of child bearing age). In one embodiment, the adult is a parent, a grandparent or other adult (e.g., a teacher, a daycare provider, a health care professional, or other adult) that is physically around and exposed to children on a daily basis. In one embodiment, the subject is not an adult (e.g., is a child) but is physically around and exposed to other non-adults/children on a daily basis.

In one embodiment, immunization with an immunogenic composition of the invention reduces and/or prevents carriage of Bordetella (B. pertussis), and reduces and/or prevents transmission of pertussis. Without being bound by theory, it is believed that antibodies specific for antigens present in the immunogenic compositions of the invention prevent the entry of Bordetella into potential host cells, thus blocking this route of infection. This is particularly advantageous when the route of entry of Bordetella into the body is through oral and mucosal epithelial cells (e.g., respiratory epithelial cells). The ability to block this route of transmission prevents or slows the development of Bordetella infection in individuals to whom immunogenic compositions/vaccines of the invention have been administered, and thus also slows or prevents transmission of Bordetella between individuals. By a “neutralizing antibody” it is meant an antibody that can neutralize (eliminate, decrease or attenuate) the ability of a pathogen to initiate and/or perpetuate an infection in a host. Without being bound by theory, it is believed that the neutralizing antibodies described herein do so by preventing (e.g. eliminating, or at least decreasing or attenuating) the ability of Bordetella to enter cells (e.g. respiratory epithelial cells).

Thus, in another embodiment, since even unimmunized subjects must acquire pertussis from others, an immunogenic composition/vaccine of the invention that reduces carriage reduces infections in immunocompromised subjects, immune-deficient subjects, subjects with immature immune systems, as well as unimmunized patients. In fact, in one embodiment, wherein an aggressive immunization program is pursued, optionally coupled with antibiotic treatment of demonstrated carriers, the invention provides the ability to eliminate or largely eliminate the human reservoir of this organism (e.g., as had been attained in the mid to late 1990's using intramuscular immunization with the cellular vaccine). Accordingly, the ability of an immunogenic composition/vaccine of the invention to protect against Bordetella (B. pertussis), colonization, as provided herein, makes possible methods to protect against disease not only in the immunized subject but, by eliminating carriage among immunized individuals, the Bordetella pathogen and any disease it causes may be eliminated from the population as a whole. Data generated during development of embodiments of the invention has documented that intranasal immunization using an immunogenic composition of the invention generates Th17 immune responses, together with Th1 type immune responses, that are important for prevention of Bordetella colonization and thus carriage (See Example 1). While an understanding of a mechanism of action is not needed to practice the present invention, and while the present invention is not limited to any particular mechanism, in one embodiment, carriage is interfered with by immunity (e.g., mucosal immunity (e.g., generation of antibodies (e.g., IgA antibodies) specific for Bordetella antigens (e.g., those required for colonization))). Again, while an understanding of a mechanism of action is not needed to practice the present invention, and while the present invention is not limited to any particular mechanism, in one embodiment, anti-Bordetella antibodies are effective against carriage in a number of ways including, but not limited to, acting at the mucosal surface by opsonizing Bordetella species thereby preventing attachment or surface invasion; and/or acting via opsonophagocytosis and killing. Vaccine compositions which are administered intranasally as provided herein may be formulated in any convenient manner and in a dosage formulation consistent with the mode of administration and the elicitation of a protective response. The quantity of antigen to be administered depends on the subject to be immunized and the form of the antigen. Precise amounts and form of the immunogenic composition (e.g., antigens) to be administered depend on the judgement of the practitioner. However, suitable dosage ranges are readily determinable by those skilled in the art and may be of the order of micrograms to milligrams. Suitable regimes for initial administration and booster doses also are variable, but may include an initial administration followed by subsequent administrations.

In some embodiments of the invention, the compositions of the invention are administered to a subject who is at risk of or likely to experience Bordetella (e.g., B. pertussis) exposure, or who is known or likely to have been or exposed, but has not yet developed infection (e.g., pertussis or whooping cough). However, in other embodiments, the composition is administered to individuals who have already developed an infection, in order to curtail the extent of infection in the individual and hasten recovery, and/or to prevent transmission to others.

As described herein, the amount of antigen in each vaccine dose is selected as an amount which induces an immunoprotective response without significant, adverse side effects in typical vaccines. Such amount will vary depending upon which specific immunogen is employed and how it is presented. The protein content of the vaccine will typically be in the range 1-100 μg, preferably 5-50 μg, most typically in the range 10-25 μg. Generally, when polysaccharides are used, it is expected that each dose will comprise 0.1-100 μg of polysaccharide where present, preferably 0.1-50 μg, preferably 0.1-10 μg, of which 1 to 5 μg is the most preferable range.

Although the vaccines of the present invention may be administered by any route, administration of the described vaccines intranasally form a preferred embodiment of the present invention.

Another preferred embodiment of the invention is a method of preventing or treating Bordetella (B. pertussis) infection or disease comprising the step of administering the immunogenic composition or vaccine of the invention to a patient in need thereof.

Another preferred embodiment of the invention is a method of preventing or treating Bordetella (B. pertussis) infection or disease comprising the step of administering the immunogenic composition or vaccine of the invention to a population (e.g., a population of families, students, health care workers, child care providers, etc.) in need thereof (e.g., in order to prevent transmission and or carriage of Bordetella (B. pertussis) within the population).

A further preferred embodiment of the invention is a use of the immunogenic composition of the invention in the manufacture of a vaccine for treatment or prevention of Bordetella (B. pertussis) infection or disease.

The term ‘Bordetella infection’ encompasses infection caused by Bordetella pertussis and other Bordetella strains capable of causing infection in a mammalian, preferably human host.

The terms “comprising”, “comprise” and “comprises” herein are intended by the inventors to be optionally substitutable with the terms “consisting of”, “consist of” and “consists of”, respectively, in every instance.

The invention is further described by reference to the following examples, which are provided for illustration only. The invention is not limited to the examples, but rather includes all variations that are evident from the teachings provided herein. All publicly available documents referenced herein, including but not limited to U.S. patents are specifically incorporated by reference.

### EXAMPLES

The following examples serve to illustrate certain preferred embodiments and aspects of the present invention and are not to be construed as limiting the scope thereof.

In the experimental disclosure which follows, the following abbreviations apply: eq (equivalents); μ (micron); M (Molar); μM (micromolar); mM (millimolar); N (Normal); mol (moles); mmol (millimoles); pmol (micromoles); nmol (nanomoles); g (grams); mg (milligrams); μg (micrograms); ng (nanograms); L (liters); ml (milliliters); μl (microliters); cm (centimeters); mm (millimeters); μm (micrometers); nM (nanomolar);° C. (degrees Centigrade); and PBS (phosphate buffered saline).

### Example 1

### Generation and Characterization of an Immunogenic Composition Comprising Nanoemulsion and B. pertussis Antigens

W805EC Nanoemulsion. W805EC, described herein, was manufactured by high-speed emulsification from ingredients that are generally recognized as safe (GRAS) with a cationic surfactant, cetylpyridinium chloride (CPC).

Vaccine preparation. The aP/NE vaccine for intranasal (IN) immunization was prepared by mixing pertussis toxin (Ptx), filamentous hemagglutinin (FHA) and pertactin (Ptn) with NE in a final concentration of NE of 20%. Conventional intramuscular (IM) vaccine was prepared by mixing all three antigens with and aluminum hydroxide gel (ALHYDROGEL) containing 2% aluminum hydroxide. Both the acellular intranasal (IN) vaccine, and the conventional acellular intramuscular vaccine, contained 4 μg Pertussis toxin (Ptx), 4 μg filamentous hemagglutinin (FHA) and 2 μg pertactin (Ptn).

ELISA. Production of specific antibodies against Ptx, Prn, and FHA were assayed using ELISA. Plates were coated with the aforementioned proteins overnight at 2-8° C. Animal sera were diluted and incubated in 96- well plates, and then following washing, HRP-conjugated secondary antibodies were added. Enhanced K-blue TMB substrate was used for color development. The optical density (OD) values were plotted against dilutions and linear regression curves were generated. Any OD value greater than 2.599 was omitted. The area under the curve was measured and IgG was calculated by comparison to the reference control. The reference control is assigned a unit value and the results were compared to that value and expressed as ELISA units (EU). In some studies, the Zollinger method was used to estimate the amount of the specific IgG in μg/ml of the reference serum. Test sera were compared to the reference sera and its immunoglobulin content was calculated in μg/ml.

Bactericidal activity. For assessing the bactericidal activity, the test sera were heat inactivated at 56° C. for 45 minutes and serial dilutions were prepared in Stainer-Scholte broth. A mixture of the test sera was added to 20% Guinea pig serum to provide the complement components, and was mixed with B. pertussis inoculum at 10⁶ to 10⁷ CFU/mL concentrations. The mixture was incubated at 37° C. for one hour, and serial dilutions were plated on Burdett Gangue agar. The plates were incubated at 37° C. for 4 days. The reduction in CFUs in test samples compared to the number of CFUs in positive control (no complement) sample was used to determine bactericidal activity. B. pertussis vaccination. A total of 24 Sprague-Dawley rats were used. The vaccine routes included intranasal (IN) and intramuscular (IM) (N=8 animals/group). A non-immunized control (N=8) was used to compare immunogenicity and cytokine production. The IN vaccinated animals received the immunogenic composition comprising Ptx, FHA and Ptn in 20% nanoemulsion, while the IM vaccinated animals received Ptx, FHA and Ptn in ALHYDROGEL. The animals were vaccinated while under ketamine/xylazine anesthesia. Animals were vaccinated three times, three weeks apart.

Cytokine assays. Spleens and lymph nodes were harvested from Sprague-Dawley rats after sacrifice at the termination of the study. Single-cell suspensions in culture medium alone (control) or, cell-suspensions activated using the different antigens were studied. Cell-free supernatants were harvested after incubation at 37 ° C. for 48 hours. T cell cytokine secretion profiles were determined by LUMINEX analysis to evaluate IFN-γ, IL-2, IL-4, IL-5, IL-10, and IL-17 using a cytokine/chemokine Milliplex MAP kit (Millipore Corp.). Data are expressed in pg/ml for each cytokine, and were obtained as the difference between the detected concentration between each antigen activated and control cells.

Animal use committee. All animal research was conducted and approved by the appropriate Committee for Use and Care of Animals where the studies were performed.

Statistics. Statistical analysis was performed using GraphPad Prism software. The analysis was performed using the Mann-Whitney non-parametric test.

Immunogenicity of the Ptx, FHA and Ptn in 20% nanoemulsion vaccine versus the Ptx, FHA and Ptn in ALHYDROGEL vaccine.

Animals received a total of three vaccinations of Ptx, FHA and Ptn in 20% nanoemulsion vaccine (NE-aP vaccine) over three week intervals. Immunogenicity and serum bactericidal activity were assessed before each boost and 6 weeks after the last dose. The Ptx, FHA and Ptn in ALHYDROGEL intramuscular vaccine (alum-aP IM vaccine) was used as a positive control.

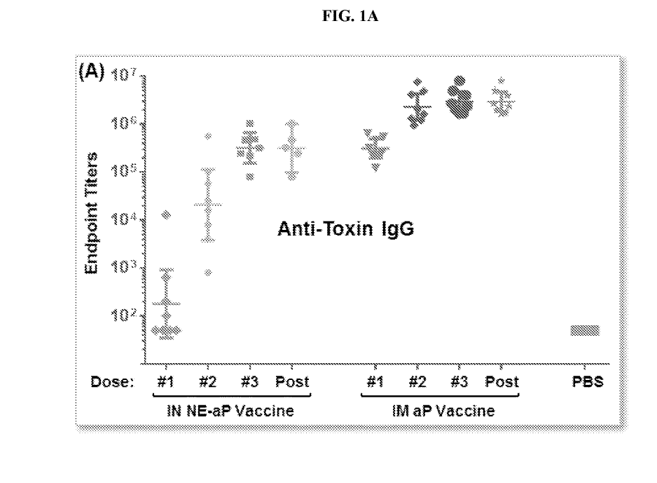

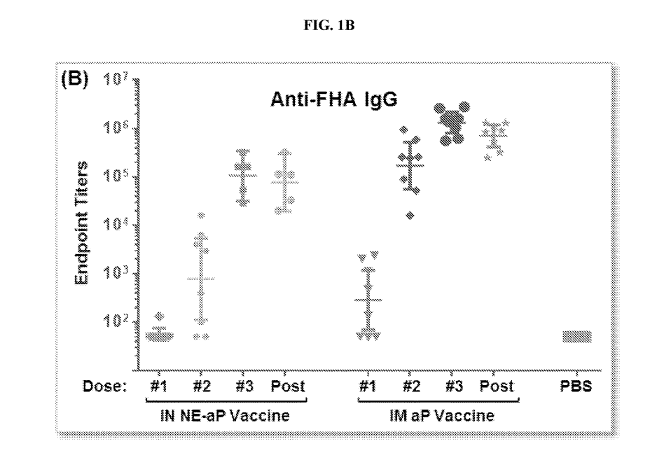

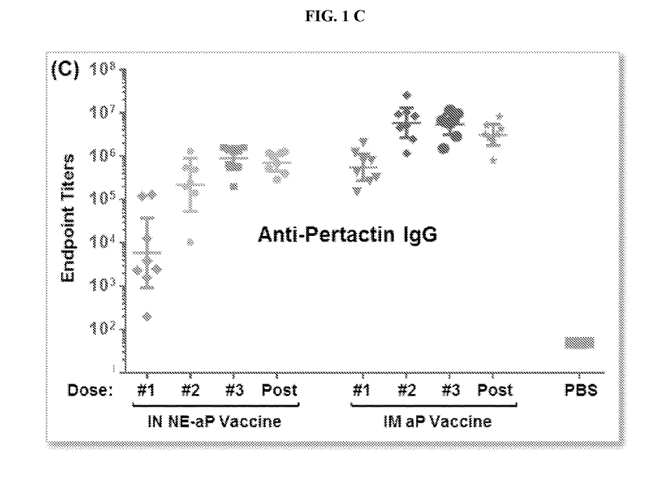

Intranasal vaccination with NE-aP vaccine elicited high levels of antibody (measured by ELISA) against all three components of the vaccine, as shown in the FIG. 1.

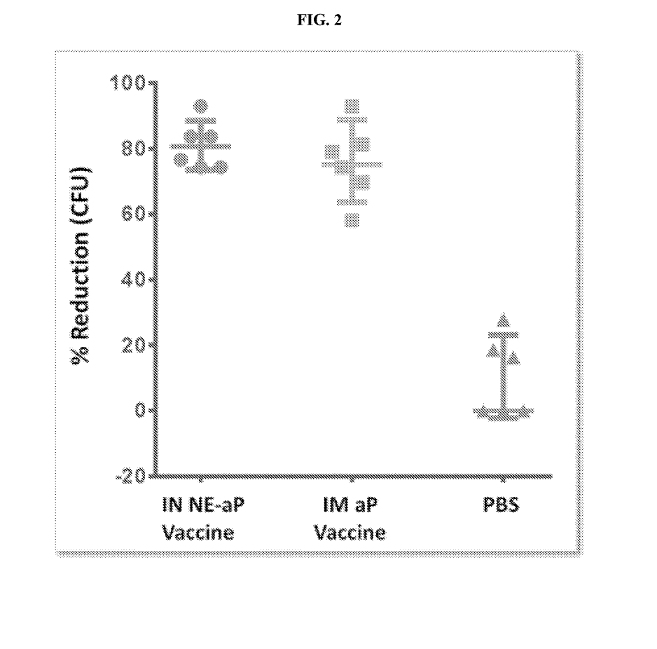

Sera from the vaccinated animals were tested for the bactericidal activity at six weeks after the third dose, as an immunological correlate of vaccine protection. As shown in FIG. 2, animals vaccinated intranasally with the NE-aP vaccine showed bactericidal activity comparable to the alum-aP IM vaccine, despite a somewhat lower level of antibodies (See FIG. 1).

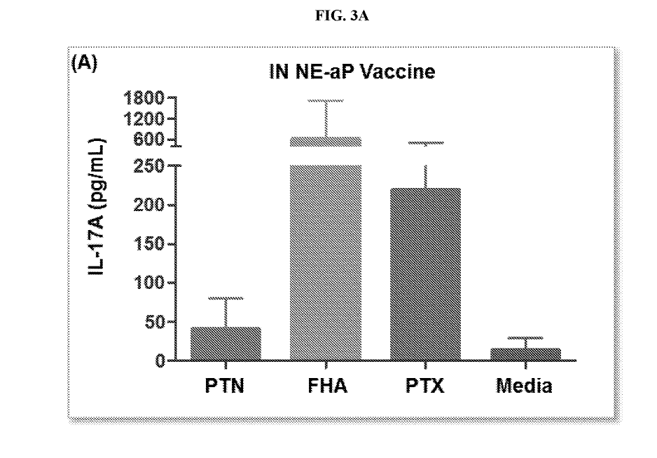

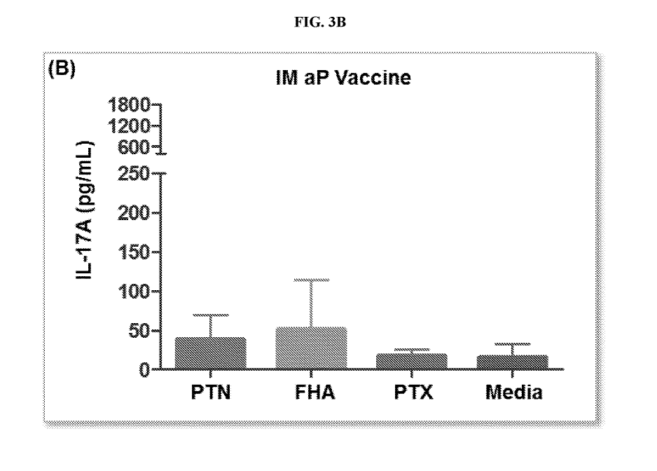

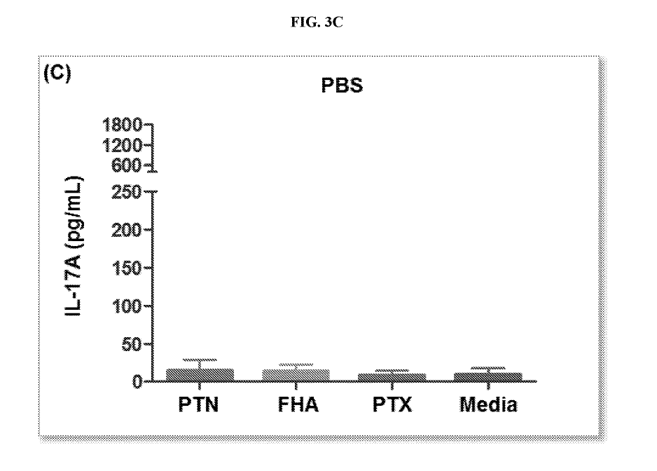

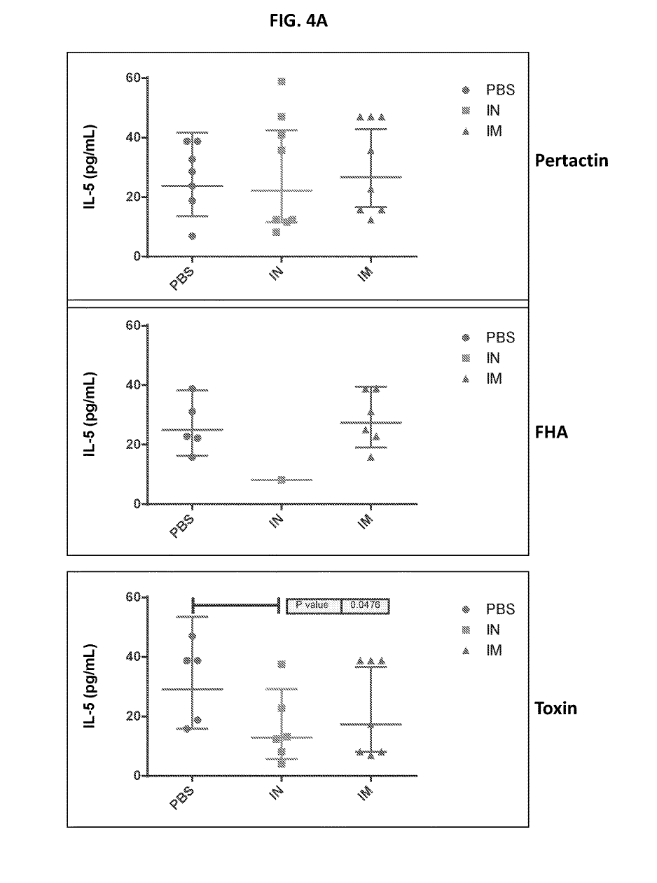

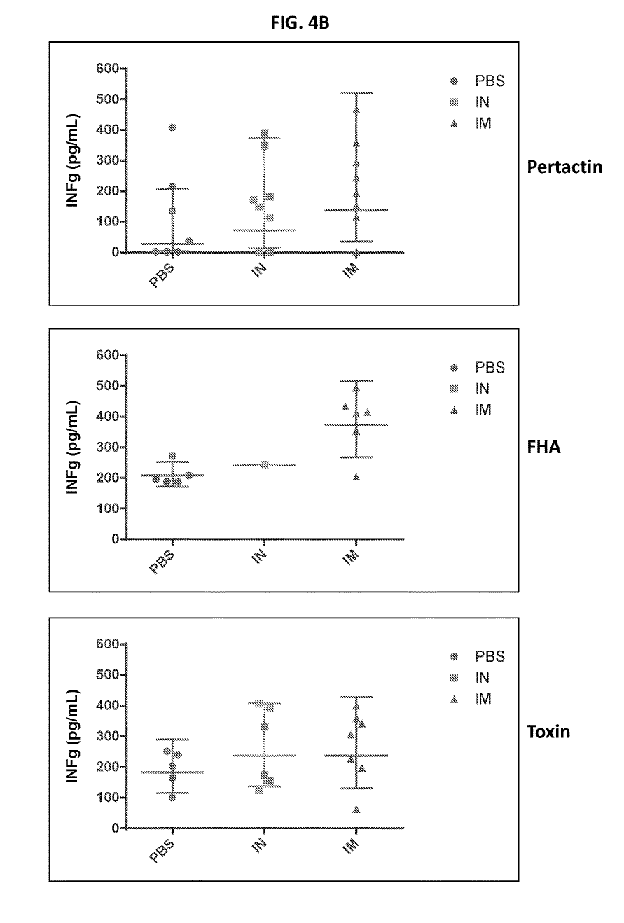

Mucosal immunity and cytokine secretion. LUMINEX multiplex analysis kits were used to evaluate mucosal immunity elicited by the intranasal NE-aP vaccine. As shown in FIG. 3, a strong IL-17 response was elicited by the NE-aP vaccine against the FHA, ptx, and to a lesser extent against Ptn (See FIG. 3A). In sharp contrast, low or negligible IL-17 responses observed using the alum-aP IM vaccine and PBS controls (See FIGS. 3B and 3C).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A method for eliciting an immunological response in a host susceptible to Bordetella pertussis carriage against colonization of B. pertussis in the nasopharynx of the host, comprising intranasally administering to the host an immunizing amount of a composition comprising:
(i) a nanoemulsion, or a dilution thereof, wherein the nanoemulsion comprises:
a) a poloxamer surfactant or polysorbate surfactant;
b) an organic solvent;
c) a halogen containing compound;
d) oil, and
e) water; and
(ii) one or more B. pertussis antigens selected from the group consisting of:
a) isolated filamentous hemagglutinin (FHA) or an immunogenic fragment thereof;
b) isolated pertactin (Ptn) or an immunogenic fragment thereof; and
c) isolated pertussis toxin (Ptx) or an immunogenic fragment thereof.

2. The method of claim 1, wherein the nanoemulsion comprises:
a) about 3 vol. % to about 15 vol. % of a poloxamer surfactant or polysorbate surfactant;
b) about 3 vol. % to about 15 vol. % of an organic solvent;
c) about 0.5 vol. % to about 1 vol. % of a halogen-containing compound;
d) about 3 vol. % to about 90 vol. % of an oil; and
e) about 5 vol. % to about 60 vol. % of water.

3. The method of claim 1, wherein the immunological response comprises induction of a Th-17 type immune response.

4. A method for eliciting a B. pertussis-specific Th-17 immune response in a host susceptible to B. pertussis carriage comprising mucosally administering to the host an effective amount of a composition comprising:
(i) a nanoemulsion, or a dilution thereof, wherein the nanoemulsion comprises:
a) about 3 vol. % to about 15 vol. % of a poloxamer surfactant or polysorbate surfactant;
b) about 3 vol. % to about 15 vol. % of an organic solvent;
c) about 0.5 vol. % to about 1 vol. % of a halogen-containing compound;
d) about 3 vol. % to about 90 vol. % of an oil; and
e) about 5 vol. % to about 60 vol. % of water; and
(ii) one or more B. pertussis antigens selected from the group consisting of:
a) isolated filamentous hemagglutinin (FHA) or an immunogenic fragment thereof;
b) isolated pertactin (Ptn) or an immunogenic fragment thereof; and
c) isolated pertussis toxin (Ptx) or an immunogenic fragment thereof;
to induce a B. pertussis-specific Th-17 immune response in the host.

5. The method of claim 4, wherein the B. pertussis-specific Th-17 immune response reduces or eliminates B. pertussis carriage in the host.

6. The method of claim 5, wherein reduction or elimination of B. pertussis carriage in the host prevents B. pertussis disease in the host.

7. The method of claim 5, wherein reduction or elimination of B. pertussis carriage in the host prevents the host from transmitting B. pertussis to another host.

8. (canceled)

9. An immunogenic composition comprising a nanoemulsion, or a dilution thereof, and at least two different proteins or immunogenic fragments thereof, wherein the at least two different proteins or immunogenic fragments thereof are selected from at least two of the following groups:
Group a)—at least one B. pertussis extracellular component binding protein or immunogenic fragment thereof selected from the group consisting of filamentous hæmagglutinin adhesin (FHA) and fimbriae;
Group b)—at least one B. pertussis transporter protein or immunogenic fragment thereof selected from the group consisting of pertactin (PRN), Vag8, BrkA, SphB1, and Tracheal colonization factor (TcfA), and
Group c)—at least one B. pertussis regulator of virulence, toxin or immunogenic fragment thereof selected from the group consisting of pertussis toxin (PT), adenylate cyclase (CyaA), Type III secretion, dermonectrotic toxin (DNT), and Tracheal cytotoxin (TCT).

10. The immunogenic composition of claim 9 comprising at least one protein or immunogenic fragment thereof from each of Group a), Group b) and Group c).

11. The immunogenic composition of claim 10, comprising isolated filamentous hemagglutinin (FHA) or an immunogenic fragment thereof of Group a); isolated pertactin (Ptn) or an immunogenic fragment thereof of Group b); and isolated pertussis toxin (Ptx) or an immunogenic fragment thereof of Group c).

12. The immunogenic composition of claim 9, wherein the nanoemulsion comprises:
a) a poloxamer surfactant or polysorbate surfactant;
b) an organic solvent;
c) a halogen containing compound;
d) oil, and
e) water.

13. The immunogenic composition of claim 9, wherein the nanoemulsion comprises:
a) about 3 vol. % to about 15 vol. % of a poloxamer surfactant or polysorbate surfactant;
b) about 3 vol. % to about 15 vol. % of an organic solvent;
c) about 0.5 vol. % to about 1 vol. % of a halogen-containing compound;
d) about 3 vol. % to about 90 vol. % of an oil; and
e) about 5 vol. % to about 60 vol. % of water.

14-16. (canceled)

17. The method of claim 1, wherein the composition comprising one or more B. pertussis antigens comprises (FHA) or an immunogenic fragment thereof, Ptn or an immunogenic fragment thereof, and Ptx or an immunogenic fragment thereof.
